(19) United States
(12) Patent Application Publication
(10) Pub. No.: US 2017/0032089 A1
(43) Pub. Date: Feb. 2, 2017

(54) MEDICAL SUPPORT APPARATUS AND SYSTEM, AND METHOD OF OPERATING MEDICAL SUPPORT APPARATUS

(71) Applicant: FUJIFILM Corporation, Tokyo (JP)

(72) Inventor: Shoji KANADA, Tokyo (JP)

(73) Assignee: FUJIFILM Corporation, Tokyo (JP)

(21) Appl. No.: 15/221,900

(22) Filed: Jul. 28, 2016

(30) Foreign Application Priority Data
Jul. 29, 2015 (JP) 2015-149688

(51) Int. Cl. G06F 19/00; A61B 5/00

(52) U.S. Cl. CPC G06F 19/345; A61B 5/4848; G06F 19/321; G06F 19/322

(57) ABSTRACT

A medical support apparatus includes a grouping unit for grouping plural lesions of a patient body treated by an anticancer chemotherapy into plural groups according to clinical onsets of the lesions. An information generator creates therapeutic effect information of therapeutic effect of the anticancer chemotherapy for respectively the plural groups. An information provider provides the therapeutic effect information. Preferably, assuming that the anticancer chemotherapy for the patient body is performed in plural therapy sessions, the groups are defined with reference to starting dates of respectively the plural therapy sessions of the anticancer chemotherapy. The lesion is further grouped (sub-grouped) according to at least one of a body organ where the lesion has occurred, and a lesion type of the lesion.

### CROSS-REFERENCE TO RELATED APPLICATION

This application claims priority under 35 USC 119 from Japanese Patent Application No. 2015-149688, filed 29 Jul. 2015, the disclosure of which is incorporated by reference herein.

### BACKGROUND OF THE INVENTION

1. Field of the Invention

The present invention relates to a medical support apparatus and system, and method of operating the medical support apparatus. More particularly, the present invention relates to a medical support apparatus and system, and method of operating the medical support apparatus, with which a doctor can perform medical care of an anticancer chemotherapy, and reliable information can be obtained for strong support in the clinical decision in making a treatment plan for the anticancer chemotherapy.

2. Description Related to the Prior Art

An anticancer chemotherapy is well-known. Assuming that a cancer of a serious stage is found in a body of a patient, anticancer drugs are administered to the patient body in the anticancer chemotherapy. U.S. Pat. Nos. 7,876,939 and 9,033,576 (corresponding to JP-A 2007-534447) disclose a technique relevant to the anticancer chemotherapy. Medical imaging of the patient body is conducted to obtain diagnostic images before and after administering the anticancer drug. Therapeutic effect of the anticancer chemotherapy is evaluated by considering a change in a size of a lesion of the cancer in the patient body.

Pharmaceutical development of the anticancer drug has been recently remarkable to increase the therapeutic effect considerably. It is possible to extend the life of the patient comparatively owing to medical care with the anticancer chemotherapy. However, it is impossible to prevent recurrence, metastasis and other problems of cancers even with the anticancer chemotherapy. The anticancer drugs are administered upon new occurrence of such problems. Also, a plurality of lesions are likely to be present simultaneously in the patient body with a time difference in a clinical onset even in the course of a period of the anticancer chemotherapy with the administration of the anticancer drugs.

It is likely that there occurs a difference in the therapeutic effect due to the number of therapy sessions of the anticancer chemotherapy. For example, the therapeutic effect for a lesion created before starting the anticancer chemotherapy is remarkable in a first therapy session, but is decreased considerably in a second therapy session. Also, it is likely that there occurs a difference in the therapeutic effect due to the elapsed time from the clinical onset of the respective lesion. For example, the therapeutic effect of the anticancer chemotherapy of a second therapy session is remarkable for a lesion shortly after the clinical onset, but is decreased considerably for a lesion much later than the clinical onset.

In case plural lesions of which the clinical onset is different are present in the patient body, the anticancer chemotherapy is a first therapy session for a first one of the lesions but may be a second therapy session for a second one of those. The number of therapy sessions of the anticancer chemotherapy may differ between the lesions. It is likely that a considerable interval of time occurs between lengths of the elapsed time from the clinical onset of the respective lesions. It is supposed that the therapeutic effect may differ between the plural lesions with the difference in the clinical onset.

U.S. Pat. Nos. 7,876,939 and 9,033,576 (corresponding to JP-A 2007-534447) discloses evaluation of the therapeutic effect without consideration of the clinical onset. A doctor must perform decision of as treatment plan of the anticancer chemotherapy with difficulty for appropriate medical care. He or she must predict various differences in the therapeutic effect according to differences in the clinical onset even by referring to the therapeutic effect technically obtained by the medical support method in relation to the treatment of plural lesions. There is no known technique for predicting differences in the therapeutic effect according to differences in the clinical onset, so that clinical decision support systems of known constructions are insufficient for supporting medical care of the doctor.

### SUMMARY OF THE INVENTION

In view of the foregoing problems, an object of the present invention is to provide a medical support apparatus and system, and method of operating the medical support apparatus, with which a doctor can perform medical care of an anticancer chemotherapy, and reliable information can be obtained for strong support in the clinical decision in making a treatment plan for the anticancer chemotherapy.

In order to achieve the above and other objects and advantages of this invention, a medical support apparatus includes a grouping unit for grouping plural lesions of a patient body treated by an anticancer chemotherapy into plural groups according to clinical onsets of the lesions. An information generator creates therapeutic effect information of therapeutic effect of the anticancer chemotherapy for respectively the plural groups. An information provider provides the therapeutic effect information.

Preferably, assuming that the anticancer chemotherapy for the patient body is performed in plural therapy sessions, the groups are defined with reference to starting dates of respectively the plural therapy sessions.

Preferably, the plural groups include a pretreatment group for grouping of a lesion of which a clinical onset is earlier than a starting date of a first therapy session among the therapy sessions, and a treatment stage group for grouping of a lesion of which a clinical onset is simultaneous with or later than the first therapy session and in a period from a starting date of a current therapy session until a date one day before a starting date of a therapy session next to the current therapy session.

Preferably, the lesions are further grouped according to at least one of a body organ of occurrence of the lesions, and a lesion type of the lesions.

Preferably, a condition of grouping of the groups is variable by an input action of manual operation.

Preferably, furthermore, a manager manages stored information of the lesions, the groups, an anticancer drug and the therapeutic effect information in an associated manner.

Preferably, furthermore, a searcher selectively reads out a common symptom image among plural diagnostic images, wherein the common symptom image is an image of one patient body having a symptom similar to a symptom of the patient body, and is an image of a lesion characteristically similar to a lesion in a patient image of the patient body. The searcher performs the readout by referring to one of the groups mapped to the lesion in the patient image and one of the groups mapped to the lesion in the common symptom image.

Preferably, the therapeutic effect information includes at least one of a change in a size of the lesions with time, a reduction factor of the size of the lesions, and a progression-free period from stabilization of the lesions after the anticancer chemotherapy until exacerbation of the lesions.

Preferably, the information provider includes a page generator for generating a page view of the therapeutic effect information. An output control unit controls an output of the page view.

Also, a method of operating a medical support apparatus includes a step of grouping plural lesions of a patient body treated by an anticancer chemotherapy into plural groups according to clinical onsets of the lesions. Therapeutic effect information of therapeutic effect of the anticancer chemotherapy is created for respectively the plural groups. The therapeutic effect information is provided.

Also, a medical support system includes a medical support apparatus, and a client terminal apparatus connected to the medical support apparatus by network connection. A grouping unit groups plural lesions of a patient body treated by an anticancer chemotherapy into plural groups according to clinical onsets of the lesions. An information generator creates therapeutic effect information of therapeutic effect of the anticancer chemotherapy for respectively the plural groups. An information provider provides the therapeutic effect information.

Consequently, reliable information can be obtained for strong support in the clinical decision in making a treatment plan for the anticancer chemotherapy, because plural lesions of a cancer can be grouped to facilitate understanding therapeutic effect of anticancer chemotherapies.

### BRIEF DESCRIPTION OF THE DRAWINGS

The above objects and advantages of the present invention will become more apparent from the following detailed description when read in connection with the accompanying drawings, in which:

FIG. 1 is a block diagram schematically illustrating a medical support system;

FIG. 2 is a data chart illustrating information in an EMR;

FIG. 3 is a data chart illustrating diagnostic images;

FIG. 4 is a data chart illustrating pathological information;

FIG. 5 is a timing chart illustrating clinical onsets of lesion;

FIG. 6 is a flow chart illustrating identification of lesions;

FIG. 7 is a block diagram schematically illustrating a computer constituting a client terminal apparatus and a server apparatus;

FIG. 8 is a block diagram schematically illustrating a CPU in the client terminal apparatus;

FIG. 9 is a block diagram schematically illustrating a CPU in the server apparatus;

FIG. 10 is a table illustrating a structure of an identifier table (grouping condition table);

FIG. 11 is a data chart illustrating an identifier list with a timeline;

FIG. 12 is a table illustrating a structure of a treatment history table;

FIG. 13 is a table illustrating a structure of a group table;

FIG. 14 is a table illustrating a structure of a therapeutic effect table;

FIG. 15 is a data chart illustrating mapping between lesions, groups, anticancer drugs and therapeutic effect information;

FIG. 16 is a block diagram schematically illustrating creation of treatment history information;

FIG. 17 is a data chart illustrating an example of the creation of treatment history information;

FIG. 18 is a timing chart illustrating treatment history information with a timeline;

FIG. 19 is a block diagram schematically illustrating creation of an identifier table (grouping condition table);

FIG. 20 is a block diagram schematically illustrating creation of a clinical onset table;

FIG. 21 is a data chart illustrating an example of the creation of the identifier table (grouping condition table);

FIG. 22 is a timing chart illustrating clinical onsets of lesions, treatment history of anticancer chemotherapies, and periods of the groups with the timeline;

FIG. 23 is a data chart illustrating an example of the creation of the clinical onset table;

FIG. 24 is a block diagram schematically illustrating creation of mapping information (grouping information);

FIG. 25 is a block diagram schematically illustrating creation of therapeutic effect information;

FIG. 26 is a data chart illustrating an example of the creation of the mapping information;

FIG. 27 is a data chart illustrating an example of the creation of the therapeutic effect information;

FIG. 28 is a screen view illustrating appearance of a page view;

FIG. 29 is a screen view illustrating a state of the page view for a period of a first therapy session;

FIG. 30 is a screen view illustrating a state of the page view for a period of a second therapy session;

FIG. 31 is a flow chart illustrating operation of a CPU;

FIG. 32 is a flow chart illustrating information provision of the CPU;

FIG. 33 is a table illustrating a structure of another preferred identifier table (grouping condition table);

FIG. 34 is a data chart illustrating a preferred embodiment in which an identifier table is manually variable;

FIG. 35 is a table illustrating a structure of a therapeutic effect table of a second preferred embodiment;

FIG. 36 is a table illustrating other preferred therapeutic effect information;

FIG. 37 is a data chart illustrating a progression-free period;

FIG. 38 is a block diagram schematically illustrating still another preferred embodiment with a searcher;

FIG. 39 is a block diagram schematically illustrating creation of an image group table;

FIG. 40 is a data chart illustrating an example of the creation of the image group table;

FIG. 41 is a data chart illustrating a preferred embodiment with similar image search.

### DETAILED DESCRIPTION OF THE PREFERRED EMBODIMENT(S) OF THE PRESENT INVENTION

### First Embodiment

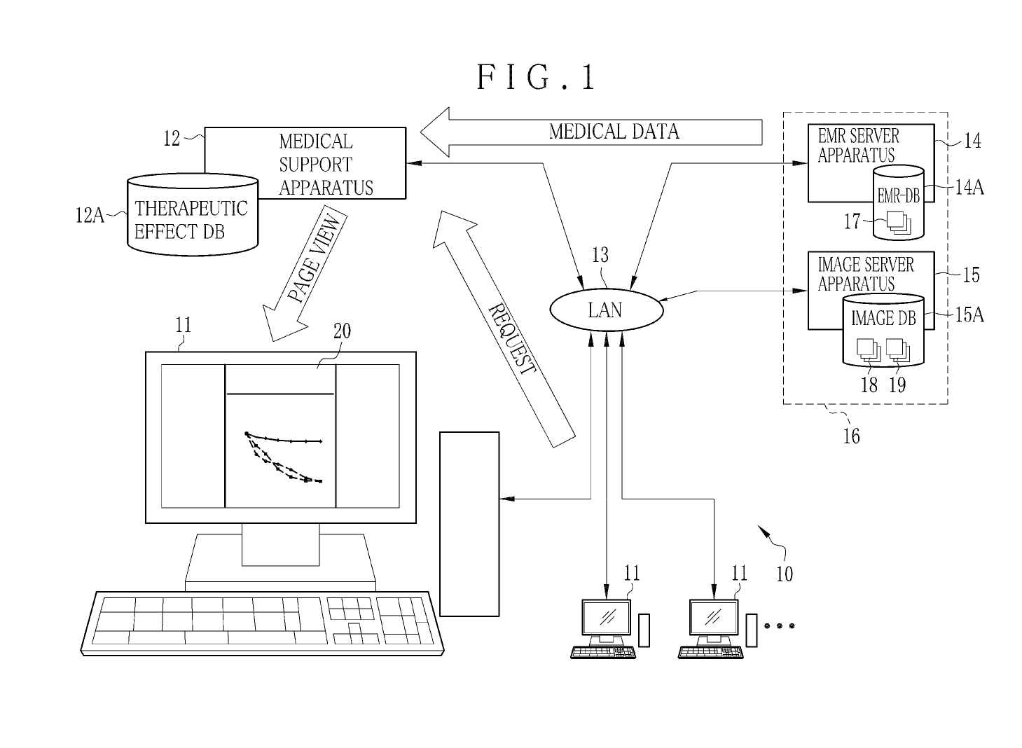

In FIG. 1, a medical support system 10 or clinical decision support system (CDSS) is installed in a medical facility, and includes a client terminal apparatus 11 or user terminal apparatus, and a medical support apparatus 12 or server apparatus. A LAN 13 or local area network as a suitable network is disposed in the medical facility, and interconnects the client terminal apparatus 11 and the medical support apparatus 12 in a communicable manner with one another.

Also, an EMR server apparatus 14 or electronic medical record server apparatus, and an image server apparatus 15 are connected with the LAN 13, and constitute a server cluster 16. An EMR database 14A or electronic medical record database is combined with the EMR server apparatus 14, and stores EMRs 17 (electronic medical records) in a searchable manner. An image database 15A is combined with the image server apparatus 15, and stores diagnostic images 18 and pathological information 19 or lesion information in a searchable manner. The diagnostic images are generated by medical imaging. The pathological information 19 is information of lesions in the diagnostic images 18.

Each of the client terminal apparatuses 11, the medical support apparatus 12 and the server cluster 16 is constituted by a computer and programs installed in the computer. Examples of the computer are a personal computer, server computer, workstation and the like. Examples of the programs are control programs such as an operating system (OS), various applications and the like.

A page view 20 of therapeutic effect information is output by the medical support apparatus 12 for therapeutic effect of an anticancer chemotherapy for administering an anticancer drug to a patient of a cancer. The EMR server apparatus 14 has a function for managing the EMRs 17. The image server apparatus 15 has a function for managing the diagnostic images 18 and the pathological information 19.

The client terminal apparatuses 11 are operated by medical professionals, such as a doctor, nurse, medical technician for diagnostic tests, and the like. A plurality of the client terminal apparatuses 11 are disposed for various medical professionals and various hospital departments, such as an internal medicine, surgery, diagnostic tests, rehabilitation, and the like. The client terminal apparatuses 11 are used while various functions of the medical support apparatus 12 and the server cluster 16 are utilized for patient care, for example, in order to view the EMRs 17, the diagnostic images 18, the pathological information 19 or the page view 20, to input various data to the EMRs 17, or to edit the pathological information 19. The client terminal apparatuses 11 can be a stationary type installed in the respective hospital departments, or a portable type manually carried by the respective medical professionals.

The medical support apparatus 12 receives a request for information distribution from the client terminal apparatus 11. Also, the medical support apparatus 12 obtains medical data (clinical data) from the server cluster 16 after acquisition in the course of medical care to the patient in the hospital facility. A therapeutic effect database 12A is combined with the medical support apparatus 12, which creates therapeutic effect information according to the obtained medical data, and writes the therapeutic effect information to the therapeutic effect database 12A. The medical support apparatus 12 creates the page view 20 according to the therapeutic effect information in the therapeutic effect database 12A. The medical support apparatus 12 transmits the page view 20 to the client terminal apparatus 11 as a requester of the request for the information distribution.

The medical support apparatus 12 distributes the page view 20 for display with a web browser. The medical support apparatus 12 issues an authorization key to the client terminal apparatus 11, and gives authorization for access to the medical support apparatus 12. The page view 20 is displayed in the client terminal apparatus 11 after reception from the medical support apparatus 12.

The medical support apparatus 12 distributes the page view 20 to the client terminal apparatus 11 in a format of XML data for web distribution created according to the XML (Extensible Markup Language) as a markup language. The client terminal apparatus 11 performs display processing to display the page view 20 on the web browser according to the XML data. Also, it is possible to use another data description language instead of the XML, such as JSON (JavaScript Object Notation) and the like, JavaScript being a trade name.

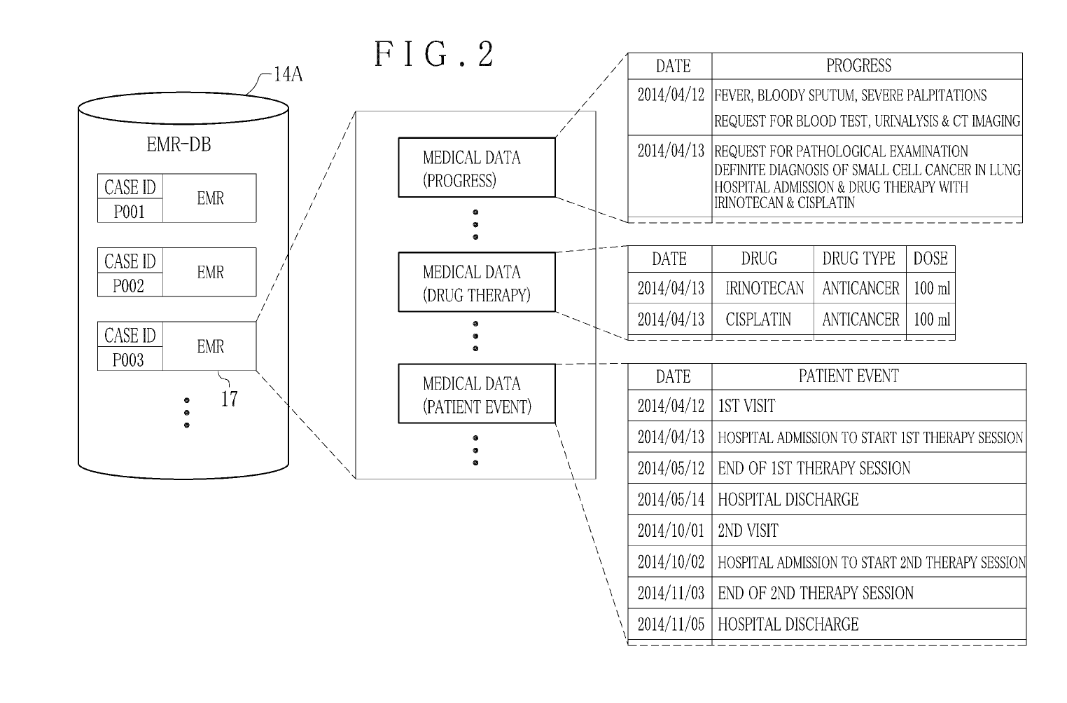

In FIG. 2, the EMRs 17 in the EMR database 14A are managed by the unit of the patients in association with a case ID (identification data) such as P001. The EMR server apparatus 14 can search the EMRs 17 from the EMR database 14A by use of the case ID as a query.

The EMRs 17 are constituted by medical data of plural data types. The data types include a progress note, drug administration and patient event. The medical data are recorded in a time sequence in a form arranged by the types. Examples of information in the progress note include talk of the patient which a doctor has heard and recorded in the EMRs 17, and medical services of medical care requested by the doctor, such as diagnostic tests, drug administration and other treatment, definite diagnosis, and the like. Examples of information of the drug administration include a drug name, drug type and dose of the administered drug. Examples of the patient events are first patient visit, second patient visit, hospital admission, hospital discharge, a start and end of an anticancer chemotherapy, and the like in the course of the treatment of the patient.

One data file of the medical data (clinical data) is constituted by calendar dates, such as a date of medical care, date of drug administration and date of a patient event, and data items, such as details of the medical care, name, type and dose of a drug, a name of the patient event and the like. Personal information of a patient (patient body) is recorded in the EMRs 17, including a case ID, name, sex, age, birthday, preference for smoking and drinking, and the like. Other examples of the medical data include measured results of vital signs, such as a heart rate, pulse rate, blood pressure, body temperature and the like, results of diagnostic tests such as a blood test, urinalysis and the like, requests for performing diagnostic tests, drafting a medical report, performing surgery and anesthesia, and the like, information of a medical history, messages and memorandum between medical professionals, and the like.

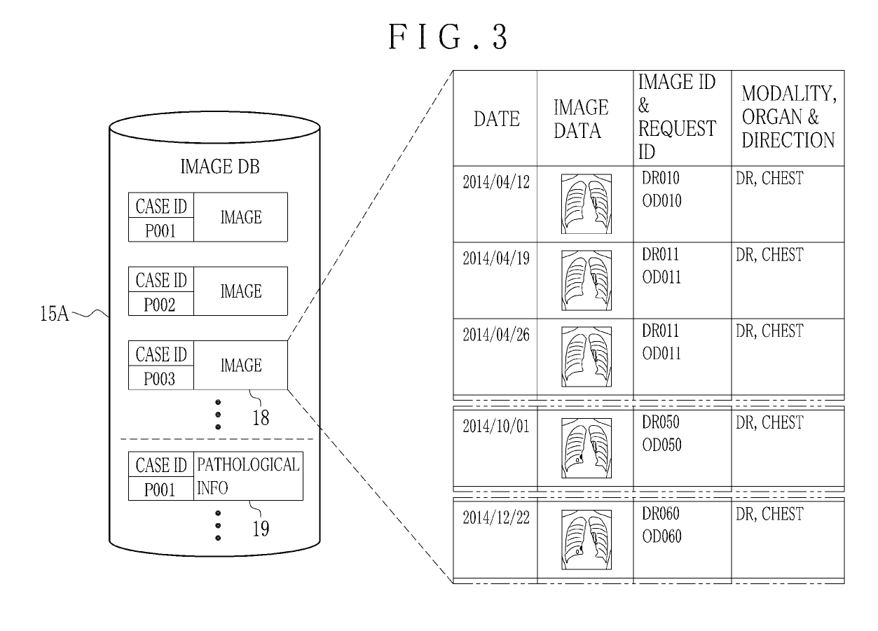

In FIG. 3, the diagnostic images 18 in the image database 15A are mapped to the case ID in the same manner as the EMRs 17, and managed by the unit of the patient. The image server apparatus 15 can search the diagnostic images 18 from the image database 15A by use of a search query of the case ID in the same manner as the EMR server apparatus 14.

The diagnostic images 18 are formed by imaging of various modalities, such as CT imaging (Computed Tomography imaging), DR imaging (Digital Radiography), ultrasound imaging (Ultrasonography or US), MRI imaging (Magnetic Resonance Imaging), endoscopic imaging and the like. For example, the diagnostic images 18 are in a format according to the DICOM (Digital Imaging and Communications in Medicine).

A data file of one case of the diagnostic images 18 is constituted by image data of the diagnostic images 18, calendar date of the imaging, image ID, request ID, modality (test type) of the imaging or diagnostic test, such as DR imaging, body part, such as the chest, direction of the imaging and the like as meta information. The image server apparatus 15 sends the diagnostic images 18 with the meta information to the medical support apparatus 12 as medical data.

An example of the image ID is a number or sign for identifying the diagnostic images 18. An example of the request ID is a number or sign for identifying a respective request for performing diagnostic imaging. For imaging of forming a plurality of diagnostic images 18 in one event of the imaging, such as CT (computed tomography), a common request ID is allocated to the set of the diagnostic images 18, which are managed together.

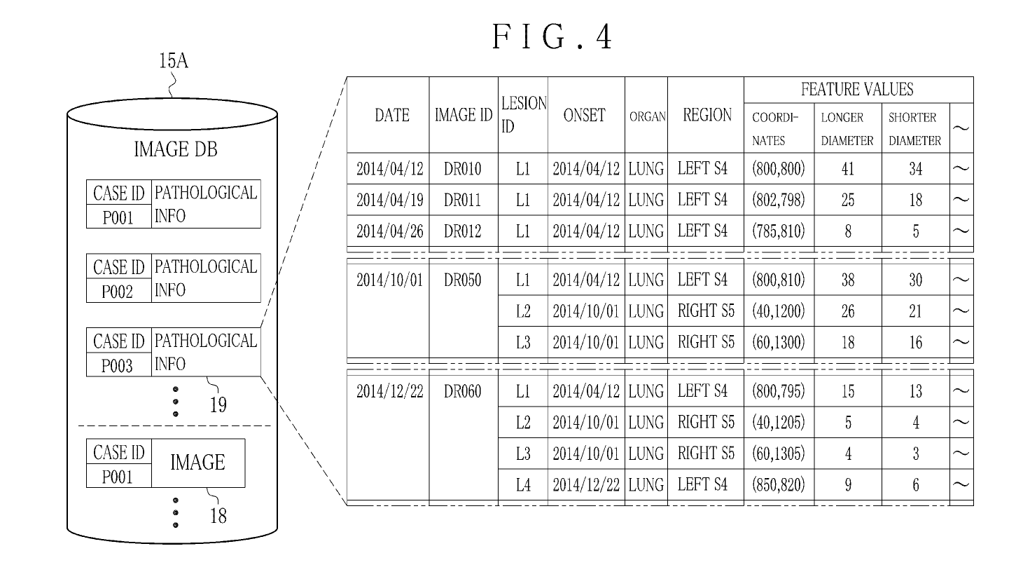

In FIG. 4, the pathological information 19 in the image database 15A is managed by the unit of the patient in association with the case ID in the same manner as the EMRs 17 and the diagnostic images 18. The image server apparatus 15 can search the pathological information 19 in the image database 15A by use of the case ID as a search query. The image server apparatus 15 transmits the pathological information 19 to the medical support apparatus 12 as medical data.

A date of performing the imaging is recorded in a data item of the pathological information 19 in the same manner as the diagnostic images 18. Also, an image ID is recorded with the pathological information 19. The image ID associates the diagnostic images 18 with the pathological information 19.

The lesion ID is alphanumeric information for recognition of the lesion in the diagnostic images 18. An example of the lesion ID is a combination of an alphabet of L and one of the numbers 1, 2, 3, 4 and so on. The number in the lesion ID expresses an order of the onset of the lesion. Thus, occurrence of a lesion of the lesion ID of L1 is the earliest. Assuming that a plurality of lesions are recorded in one of the diagnostic images 18, plural lesion IDs for the plural lesions are mapped to one image ID, for example, lesion IDs of L1, L2, L3 and so on in connection with the image ID of DR050 on the date of Oct. 1, 2014.

A recorded clinical onset (date of onset) includes a date of performing the imaging of the diagnostic images 18 and initially allocating the lesion ID, namely, a date of discovery of the lesion of the lesion ID in the diagnostic images 18. For example, the clinical onset of the lesion ID of L1 is Apr. 12, 2014. The clinical onset of the lesion ID of L4 is Dec. 22, 2014.

Information of a body organ of interest, a region of interest of the lesion, and an image feature value is recorded in a data item of the lesion ID. The information of the organ of interest and the region of interest is input by a doctor, or automatically detected by image analysis in the image server apparatus 15. Examples of the image feature value include position coordinates of a position of the lesion in the diagnostic images 18, and longer and shorter diameters of the lesion as sizes.

The position coordinates are numerical information of a location of a pixel constituting the diagnostic images 18. The position coordinates are two-dimensional position coordinates assuming that the diagnostic image 18 is a two-dimensional image, such as a DR image, and is a three-dimensional position coordinates assuming that the diagnostic image 18 is a three-dimensional image, such as a CT image. A recorded example of the position coordinates is coordinates of a center or gravity center of an area of a lesion designated by a doctor, or an area of a lesion automatically extracted by the image server apparatus 15 according to image analysis. Recorded examples of the longer and shorter diameters are diameters input by the doctor, or diameters automatically obtained by the image server apparatus 15 according to image analysis.

Further examples (not shown) of the image feature values include pixel-related feature values and shape-related feature values in addition to the position coordinates and longer and shorter diameters. The pixel-related feature values are a maximum, minimum, average and variance of the pixel values in an area of the lesion. The shape-related feature values are scores of eccentricity, convexity and roundness in the area of the lesion.

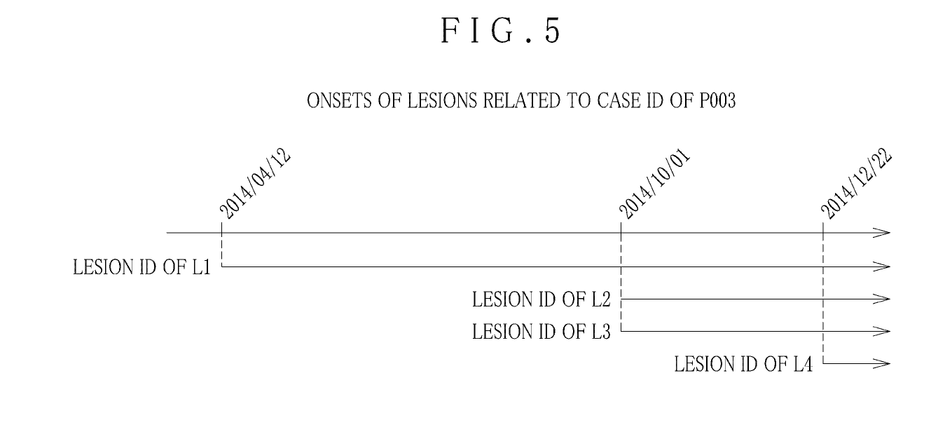

In FIG. 5, clinical onsets of lesions of the patient body of the case ID of P003 are indicated on the timeline. Four lesions of lesion IDs L1, L2, L3 and L4 have occurred to the patient body of the case ID of P003. The clinical onset of the lesion of the lesion ID of L1 is Apr. 12, 2014 which is the earliest. The clinical onset of the lesions of the lesion IDs of L2 and L3 is Oct. 1, 2014. The clinical onset of the lesion of the lesion ID of L4 is Dec. 22, 2014.

The image server apparatus 15 specifies the lesion before recording the new diagnostic image 18 to the image database 15A. Specifying the lesion is to check whether a lesion in the new diagnostic image 18 to be recorded is identical with a lesion in the past diagnostic image 18 stored in the image database 15A.

The image server apparatus 15, assuming that it is judged that a lesion in the new diagnostic image 18 is the same as a lesion in the past diagnostic image 18, a lesion ID the same as the lesion in the past diagnostic image 18 is allocated to the lesion in the new diagnostic image 18. The image server apparatus 15, assuming that it is judged that a lesion in the new diagnostic image 18 is different from the lesion in the past diagnostic image 18, a new lesion ID is allocated to the lesion in the new diagnostic image 18. Assuming that there is no relevant past-diagnostic image 18, a new lesion ID is allocated to the lesion in the new diagnostic image 18.

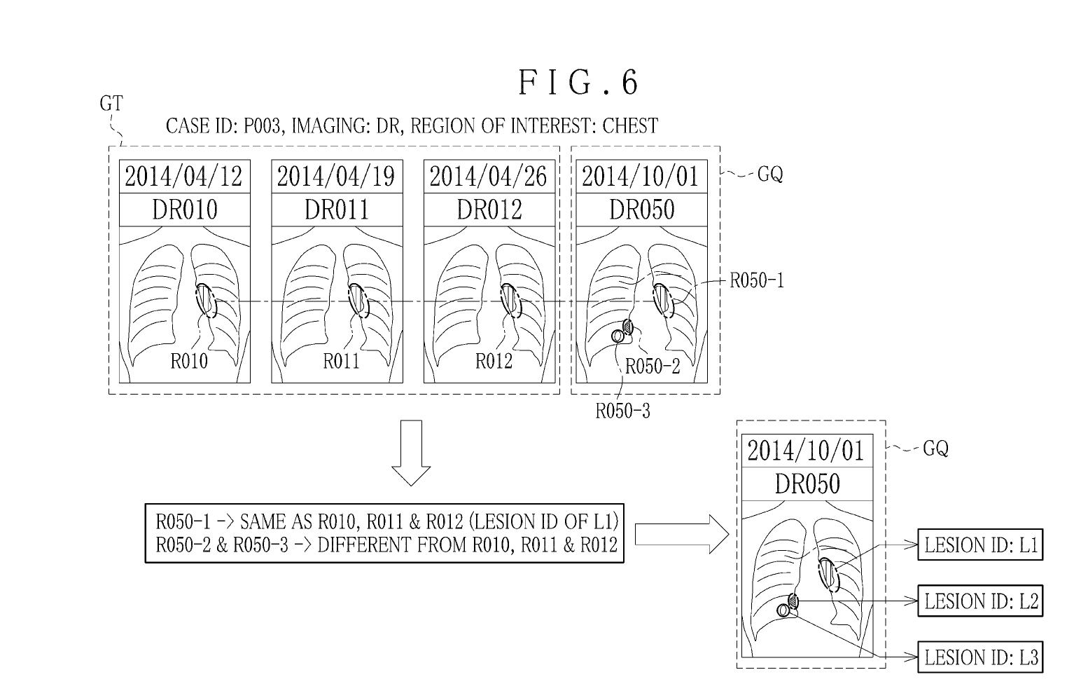

In FIG. 6, specifying the lesion in the diagnostic images 18 of the patient body of the case ID of P003 is illustrated. For example, the diagnostic image 18 indicated by the dotted lines with the sign GQ is recorded to the image database 15A, the diagnostic images 18 having the date of Oct. 1, 2014, the image ID of DR050, the modality (test type) of DR imaging, and the region of interest of the chest.

At first, the image server apparatus 15 searches the diagnostic images 18 from the image database 15A according to equality to the diagnostic image GQ in relation to the case ID, modality (test type) and the region of interest. In the embodiment, three of the diagnostic images 18 are searched and read as indicated by the dotted lines and sign GT, including an image with the date of Apr. 12, 2014 and the image ID of DR010, an image with the date of Apr. 19, 2014 and the image ID of DR011, and an image with the date of Apr. 26, 2014 and the image ID of DR012. The diagnostic images GT correspond to the diagnostic images 18 of the past described above, and are images of the same patient body, the same modality of imaging and the same region of interest as the new diagnostic image CQ, but with a different date from the diagnostic image CQ.

Then the image server apparatus 15 checks whether the areas R010, R011 and R012 of the lesion in the diagnostic image GT is the same as the areas R050-1, R050-2 and R050-3 of the lesion in the diagnostic image GQ. To this end, the image server apparatus 15 utilizes the non-rigid body positioning technique disclosed in U.S. Pat. No. 7,117,026 (corresponding to JP-A 2005-528974).

In FIG. 6, it is judged that the area R050-1 of the lesion in the image GQ is equal to the areas R010, R011 and R012 of the lesion in the image GT, and that the areas R050-2 and R050-3 of the lesion in the image GQ are different from the areas R010, R011 and R012 of the lesion in the image GT. The image server apparatus 15 allocates the lesion ID of L1 of the lesion of the areas R010, R011 and R012 to the areas R050-1. The image server apparatus 15 allocates new lesion IDs of L2 and L3 to the areas R050-2 and R050-3. It is possible to keep correctness by identifying the lesion, and prevent an error of allocating plural lesion IDs to an equal lesion, or an error of allocating an equal lesion ID to plural lesions different from one another.

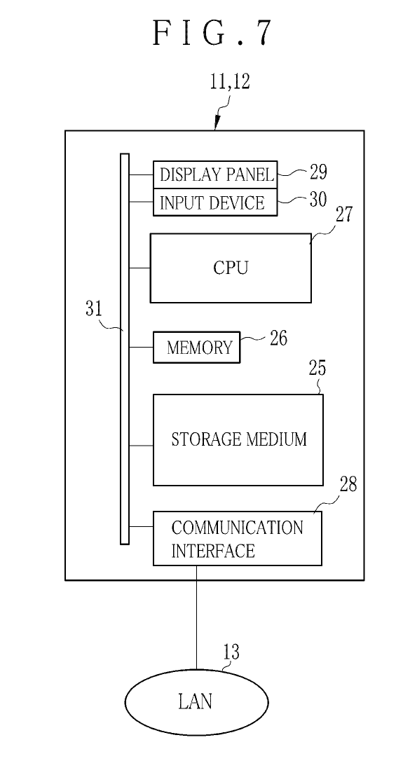

In FIG. 7, each of the client terminal apparatuses 11 and the medical support apparatus 12 is constituted by a computer of a basically equal structure, and has a storage medium 25 or storage device, a memory 26, a CPU 27 or central processing unit, a communication interface 28, a display panel 29 and an input device 30. A data bus 31 interconnects those circuit devices.

The storage medium 25 is a hard disk drive incorporated in the computer constituting the client terminal apparatus 11, or connected to the computer by a cable, network or the like. Also, the storage medium 25 may be a disk array having plural hard disk drives. The storage medium 25 stores a control program and various application programs such as the Operating System (OS), and display page data for control pages associated with the programs.

The memory 26 is a working memory with which the CPU 27 performs tasks. The CPU 27 loads the memory 26 with the programs stored in the storage medium 25, and controls the various circuit devices in the computer by performing the tasks according to the program.

The communication interface 28 is a network interface for control of transmission of various data by use of the LAN 13. The display panel 29 displays a control page where an input action can be performed by use of the input device 30, such as a mouse, keyboard and the like. The control page has a function according to the GUI (graphical user interface). The computer constituting the client terminal apparatus 11 receives manual inputs from the input device 30 by use of the various menus in the control page.

In the following description, a sign A will be added to each of reference signs of components in the computer constituting the client terminal apparatus 11. A sign B will be added to each of reference signs of components in the computer constituting the medical support apparatus 12.

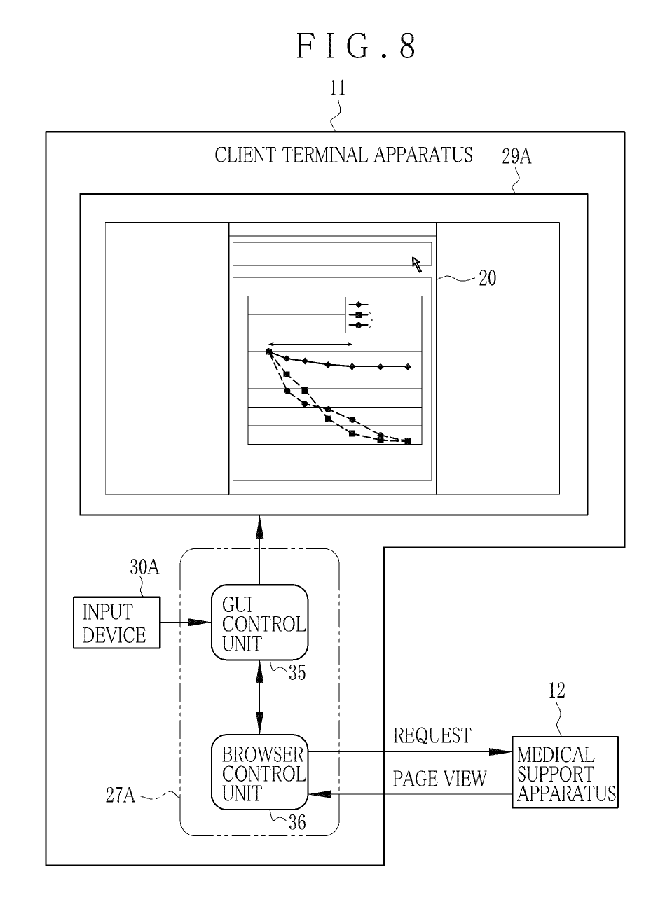

In case a web browser is run, a CPU 27A or central processing unit in the client terminal apparatus 11 in FIG. 8 cooperates with the memory 26 to function a GUI control unit 35 and a browser control unit 36 or display processor.

A display panel 29A is controlled by the GUI control unit 35 and displays various control pages or views. An input device 30A is operated to perform input actions to the GUI control unit 35 by use of a control pages or views. One example of the input actions is a request for information distribution of the page view 20 to the medical support apparatus 12. The GUI control unit 35 sends signals of the input actions to the browser control unit 36.

The browser control unit 36 controls operation of the web browser. The browser control unit 36 outputs the request for information distribution to the medical support apparatus 12 according to the command signal from the GUI control unit 35.

The browser control unit 36 receives image data of the page view 20 from the medical support apparatus 12. The browser control unit 36 forms the page view 20 according to the image data in a manner for display on the web browser, and sends the page view 20 to the GUI control unit 35. The GUI control unit 35 drives the display panel 29A to display the page view 20.

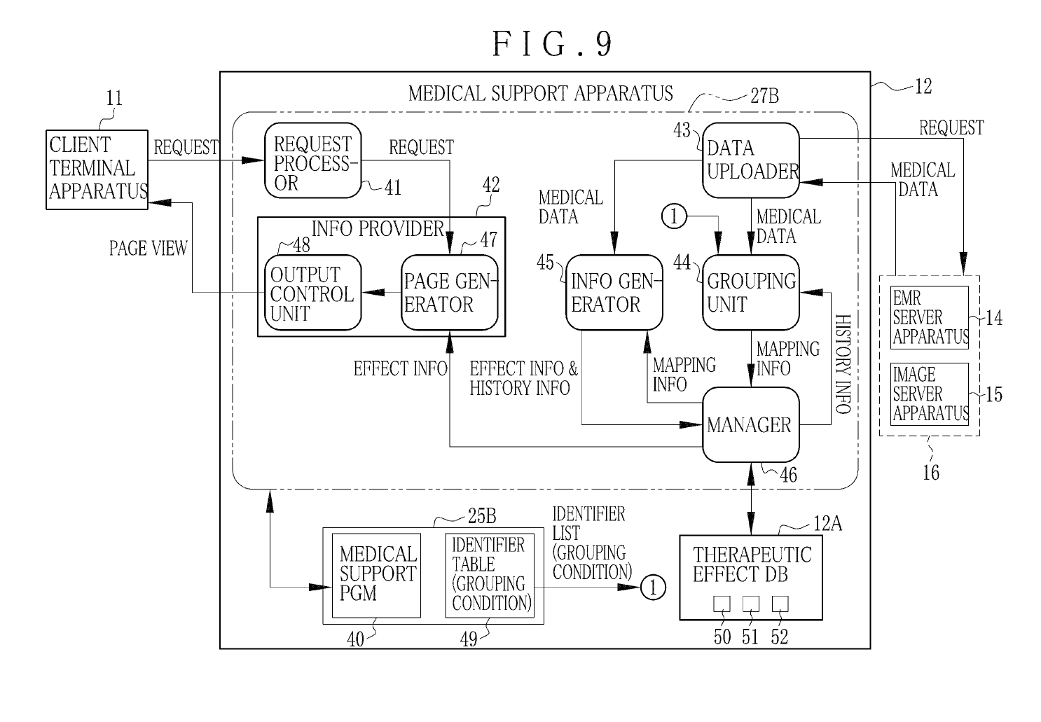

In FIG. 9, a storage medium 25B or storage device in the medical support apparatus 12 stores a medical support computer-executable program 40. The computer-executable program 40 is an application for functioning the computer for the medical support apparatus 12.

Running the computer-executable program 40 causes a CPU 27B in the medical support apparatus 12 to activate a request processor 41 or request receiving unit, an information provider 42 or notifier, a data uploader 43 or data acquisition unit, a grouping unit 44 or classifier, an information generator 45 and a manager 46. The information provider 42 is constituted by a page generator 47 and an output control unit 48 or display processor.

The request processor 41 receives a request for information distribution from the client terminal apparatus 11. The request processor 41 sends the request to the page generator 47.

The page generator 47 receives the therapeutic effect information from the manager 46 in response to the request for the information distribution, and creates the page view 20 according to the received therapeutic effect information. The page generator 47 transmits the page view 20 to the output control unit 48. The output control unit 48 outputs the page view 20 to the client terminal apparatus 11 as a requester of the request.

The data uploader 43 sends a request for acquisition of medical data to the server cluster 16 periodically, for example, one time per a day. The data uploader 43 acquires the medical data from the server cluster 16 in response to the request for the acquisition. The data uploader 43 enters the acquired medical data to the grouping unit 44 and the information generator 45.

The grouping unit 44 groups plural lesions into plural case groups according to the clinical onsets after performing the anticancer chemotherapy to the patient body. The grouping unit 44 creates mapping information (grouping information) of results of grouping, and inputs this to the manager 46.

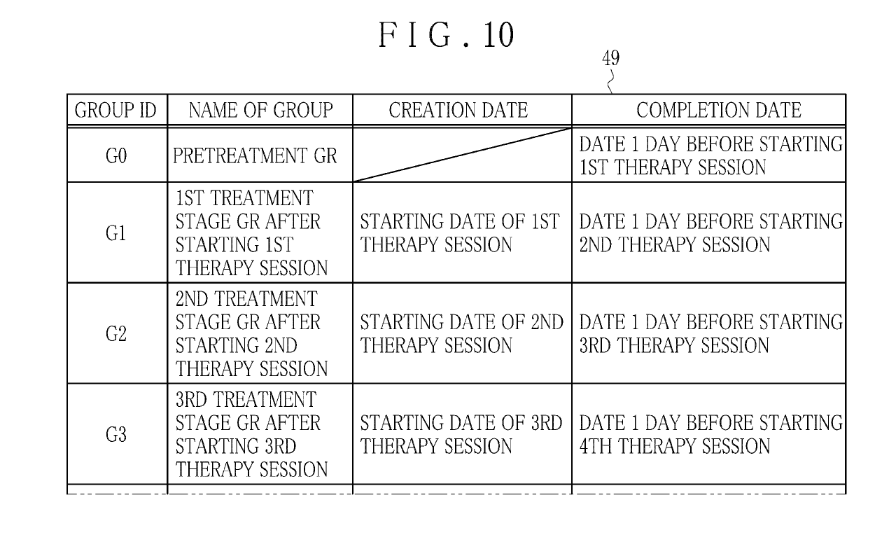

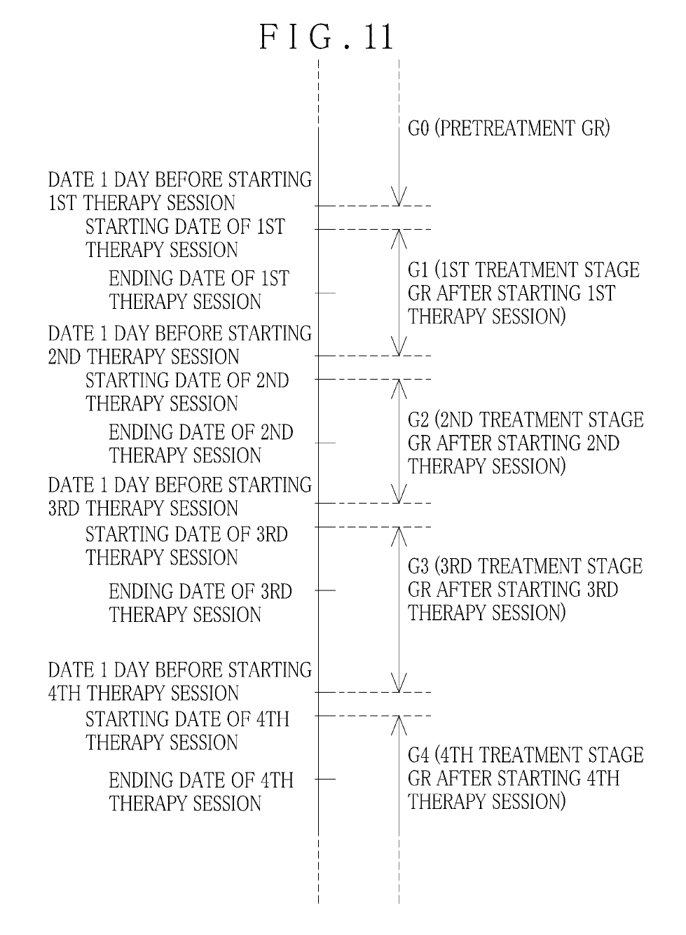

The information generator 45 creates the therapeutic effect information for the respective case groups according to the medical data from the data uploader 43 and the mapping information from the manager 46. The information generator 45 inputs the therapeutic effect information to the manager 46. The information generator 45 creates treatment history information according to medical data from the data uploader 43 for a treatment history of the anticancer chemotherapy for the patient. The information generator 45 outputs the created treatment history information to the manager 46. The grouping unit 44 performs grouping according to an identifier list (in FIGS. 10 and 11) from an identifier table 49 (grouping condition table) (in a table memory) stored in the storage medium 25B, in combination with the medical data from the data uploader 43 and the treatment history information from the manager 46.

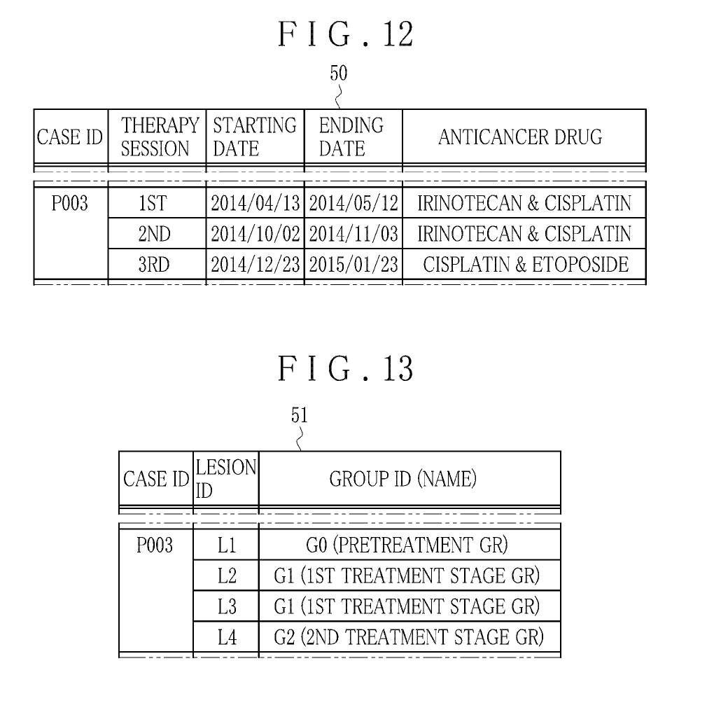

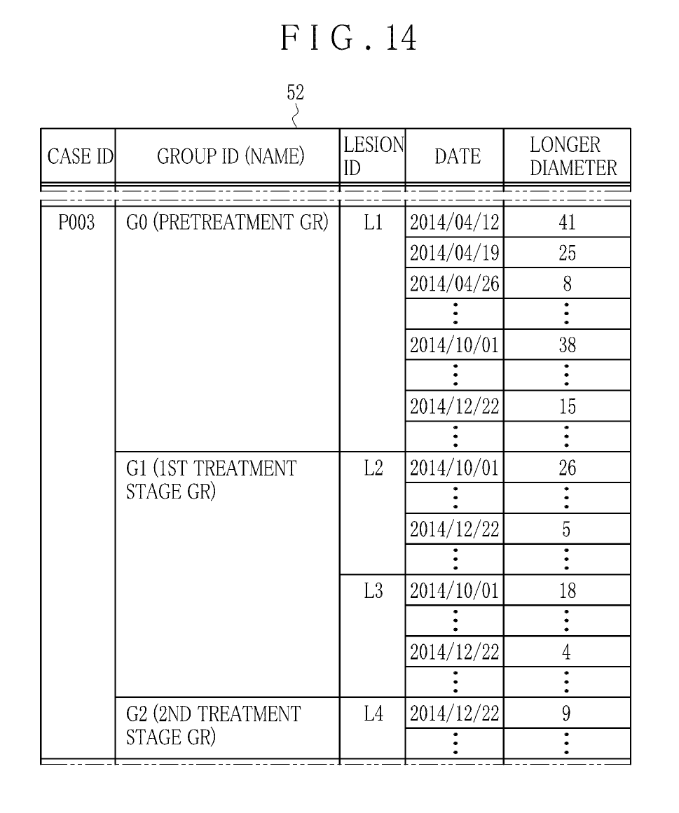

The therapeutic effect database 12A stores information of a treatment history table 50 (in a table memory), a group table 51 (grouping information table) (in a table memory) and a therapeutic effect table 52 (in a table memory). The treatment history table 50 contains registered treatment history information of FIG. 12. The group table 51 contains registered mapping information of FIG. 13. The therapeutic effect table 52 contains registered therapeutic effect information of FIG. 14. The manager 46 manages registration of information to the information tables 50-52 and readout of the information from those. For example, the manager 46 reads out the therapeutic effect information from the therapeutic effect table 52, and sets the therapeutic effect information to the page generator 47 for creating the page view 20.

In FIG. 10, the identifier table 49 contains information of group IDs, group names of the case groups, creation dates and completion dates. The group IDs are numbers or signs for recognizing plural case groups. The creation dates and completion dates are in the identifier list, and are starts and ends of the respective periods of the case groups.

Examples of the case groups are a pretreatment group (group ID of G0) before starting the first therapy session and treatment stage groups (group IDs of G1, G2 and so on) after starting the first therapy session. No information is registered for a creation date of the pretreatment group. Information of “the date one day before the starting date of the first therapy session” is registered for a completion date of the pretreatment group. Thus, a lesion (original lesion) corresponding to a period before the starting date of the first therapy session is grouped for the pretreatment group.

For a creation date of respective treatment stage groups, a starting date of a current therapy session is registered, for example, a starting date of a second therapy session for the second treatment stage group after starting the second therapy session of the group ID of G2. For a completion date of respective treatment stage groups, a date one day before a starting date of a succeeding therapy session is registered, for example, a starting date of a fourth therapy session for the third treatment stage group after starting the third therapy session of the group ID of G3. Let N be a natural number equal to or more than 1. For a creation date of respective treatment stage groups after starting an Nth therapy session, a starting date of the Nth therapy session is registered. For a completion date of the respective treatment stage groups after starting the Nth therapy session, a date one day before a starting date of an (N+1)th therapy session is registered. In each of the treatment stage groups, a lesion of a clinical onset in a period after the first therapy session is grouped, the period being from the starting date of the current therapy session to a date one day before the starting date of a succeeding therapy session.

In FIG. 11, an identifier list (grouping condition) in the identifier table 49 in FIG. 10 is indicated with a timeline. The grouping is performed according to a date of starting each therapy session of an anticancer chemotherapy. Assuming that next therapy session has not started yet, a data item of the completing the treatment stage group is automatically a date of acquiring the medical data in the data uploader 43.

In FIG. 12, registered information in the treatment history table 50 includes treatment history information for the respective case IDs. The treatment history information is constituted by a starting date and ending date of the respective therapy session and drug name of the administered anticancer drugs. In FIG. 12, only treatment history information of the patient body of the case ID of P003 is illustrated. However, treatment history information of other patient bodies is actually registered. The group table 51 in FIG. 13 and the therapeutic effect table 52 in FIG. 14 are structured similarly.

In FIG. 13, the mapping information is registered in the group table 51 for the respective case IDs. The mapping information is constituted by the lesion ID and the group ID of the case group into which the lesions of the lesion IDs are grouped by the grouping unit 44. Note that group names are added to the group IDs in FIG. 13 for facilitating understanding. The therapeutic effect table 52 in FIG. 14 is depicted in the same manner.

In FIG. 14, registered information in the therapeutic effect table 52 includes therapeutic effect information for the respective case IDs. The therapeutic effect information is arranged for the respective group IDs, and constituted by a lesion ID, date of performing the imaging, and image feature value. A relationship between the group ID and the lesion ID is equal to a relationship between the group ID and the lesion ID in the group table 51. The therapeutic effect information of one of the lesion IDs is information of a change of the longer diameter of the lesion of the lesion ID in the time sequence.

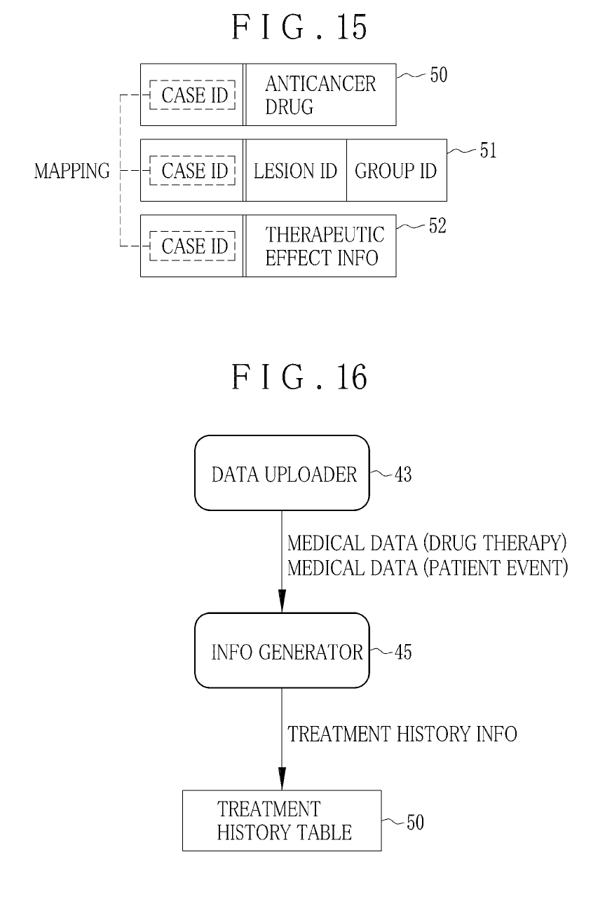

In FIG. 15, various data are associated with one another by use of the case ID, including information of the anticancer drugs in the treatment history table 50, the lesion IDs and the group IDs in the group table 51, and the therapeutic effect information in the therapeutic effect table 52. Note that the various data can be associated not with the case ID but with a specialized link ID, which can be issued by the manager 46. Also, a single table (in a table memory) can be provided as a combination of the information tables 50-52, and used for managing the various data.

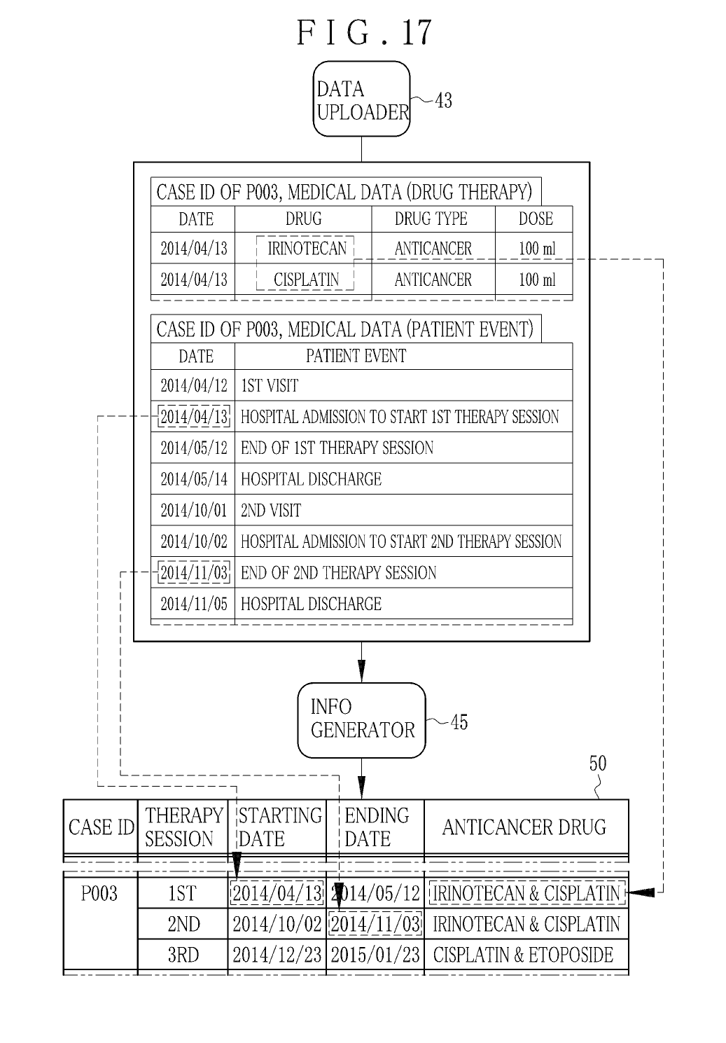

Functions of the circuit devices in the CPU 27B in the medical support apparatus 12 are described now by referring to FIGS. 16, 19, 20, 24 and 25 and examples in FIGS. 17, 18, 21-23, 26 and 27.

In FIG. 16, the information generator 45 creates the history information according to the medical data from the data uploader 43 in relation to the drug administration and patient events. Note that the manager 46 is omitted from FIGS. 16, 17, 19, 21, 24-27 for simplification.

In FIG. 17, creation of the history information of the patient body of the case ID of P003 is illustrated. The information generator 45 performs access to medical data of the patient event of the case ID of P003, and selectively reads out a starting date and an ending date of the respective therapy session, such as Apr. 13, 2014 as the starting date of the first therapy session, and Nov. 3, 2014 as the ending date of the second therapy session.

Also, the information generator 45 reads the name of the drugs administered in the anticancer chemotherapy from the medical data of the drug administration for the patient body with the case ID of P003, for example, irinotecan and cisplatin administered on Apr. 13, 2014 as a date of starting a first therapy session.

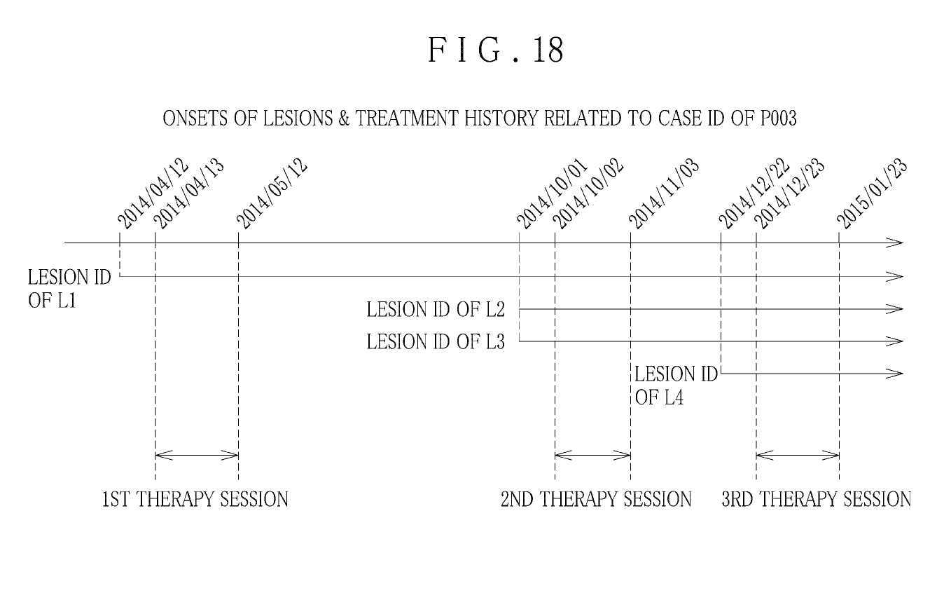

Thus, the starting date and ending date of the respective therapy session and the drug names of the anticancer drugs administered in the anticancer chemotherapy are registered to the treatment history table 50 as treatment history information after readout in the information generator 45. In FIG. 18, the treatment history of the anticancer chemotherapy of the patient body of the case ID of P003 and the period of occurrence of the lesion are indicated along a timeline.

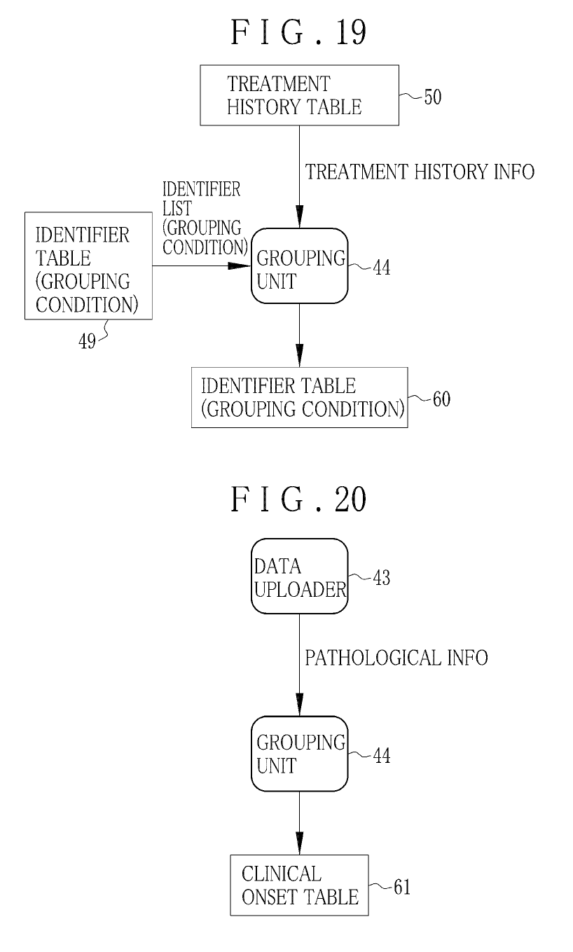

The grouping unit 44 creates two intermediate data before grouping the lesions. At first, the grouping unit 44 in FIG. 19 creates an identifier table 60 (grouping condition table) by way of the intermediate data according to the treatment history information from the treatment history table 50 and the identifier list from the identifier table 49. The identifier table 60 contains registered data of the identifier list for the respective case ID, the data being specialized for each of the patients after rewriting the identifier list in the identifier table 49 according to the treatment history information.

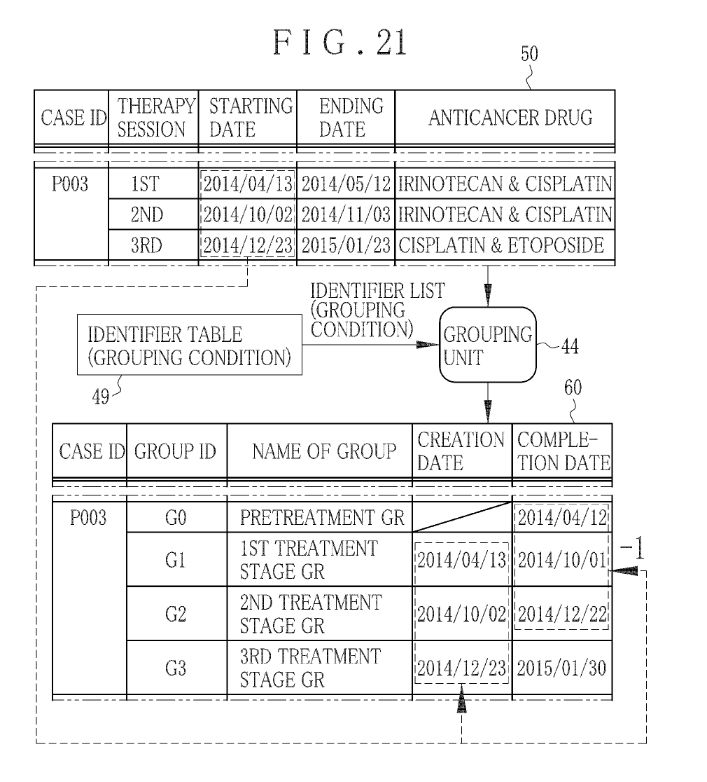

In FIG. 20, the grouping unit 44 creates a clinical onset table 61 or date of onset table as intermediate data, according to the pathological information 19 from the data uploader 43. The clinical onset table 61 contains registered information of lesion IDs in the pathological information 19 of respective patient bodies and their clinical onsets by the unit of the case IDs. The grouping unit 44 creates the identifier table 60 and the clinical onset table 61 because various data are different between patient bodies, such as the number of the therapy sessions of the anticancer chemotherapy, starting and ending dates of the therapy sessions, and clinical onsets of the respective lesions.

In FIG. 21, creation of the identifier table 60 for the patient body of the case ID of P003 is illustrated. The grouping unit 44 reads out starting dates of the respective therapy sessions, such as Apr. 13, 2014, Oct. 2, 1014 and Dec. 23, 1014 from the data item of the case ID of P003 in the treatment history table 50. The grouping unit 44 writes the starting dates to data items of the creation dates of the treatment stage groups in the data item of the case ID of P003 in the identifier table 60.

Also, the grouping unit 44 writes earlier dates Apr. 12, 2014, Oct. 1, 2014 and Dec. 22, 2014 to data items of the completion dates of the pretreatment group and first and second treatment stage groups in the data item of the case ID of P003 in the identifier table 60, the first and second treatment stage groups being after starting one and two therapy sessions. The earlier dates are dates one day before respectively the dates of starting the respective therapy sessions.

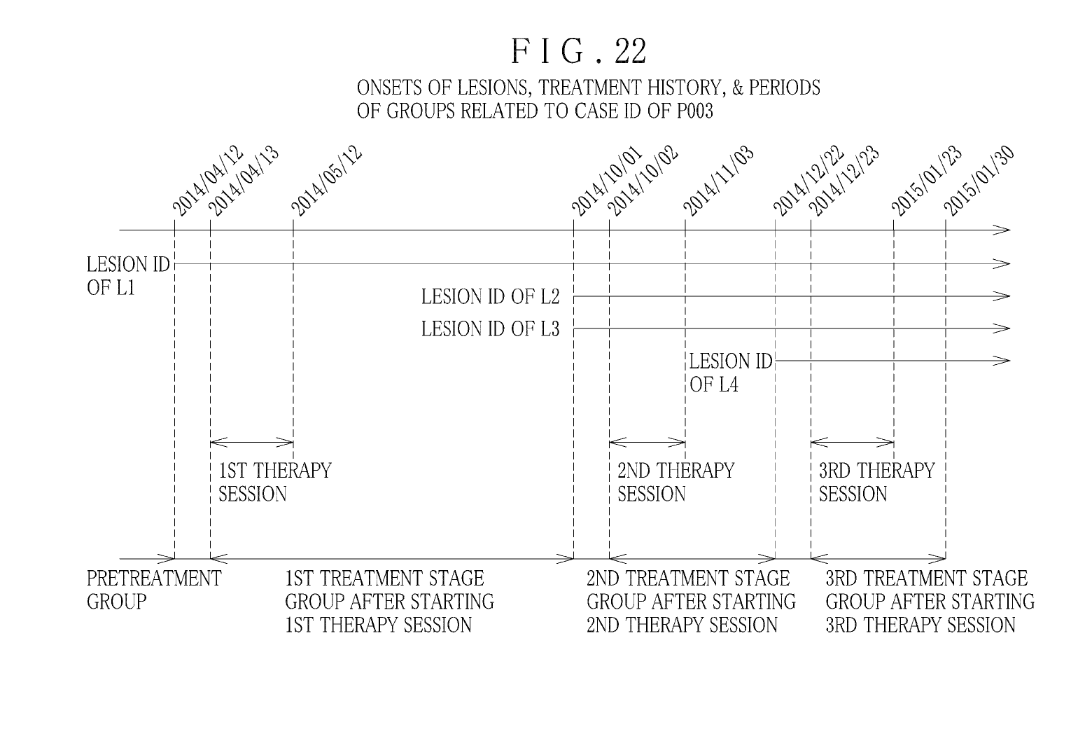

A fourth therapy session has not started yet for the patient body of the case ID of P003. A date Jan. 30, 2015 of acquiring the medical data is automatically set for a completion date of the third treatment stage group after starting three therapy sessions. In FIG. 22, a period of the respective case groups of the patient of the case ID of P003 is indicated on the timeline with the clinical onset and the treatment history of the lesion. Note that it is unnecessary to register an ending date of the respective therapy session in the treatment history table 50, as the completion date of the respective therapy session is unrelated to the identifier list.

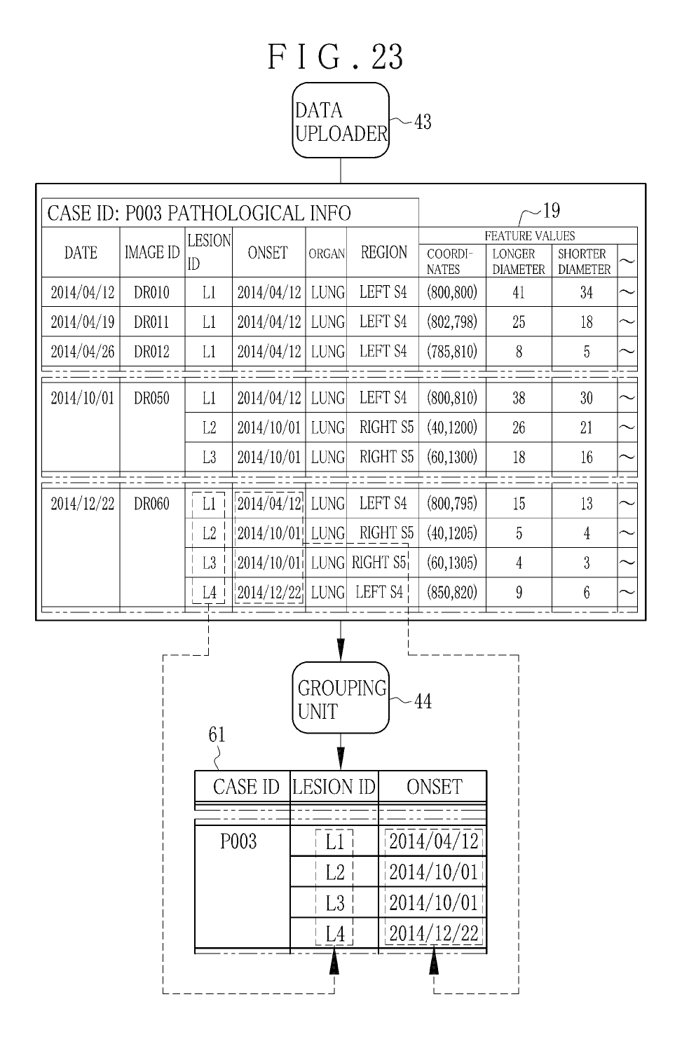

In FIG. 23, creation of the clinical onset table 61 of a patient body of the case ID of P003 is illustrated. The grouping unit 44 reads out lesion IDs of L1, L2, L3 and L4 and their associated clinical onsets of Apr. 12, 2014, Oct. 1, 2014, Oct. 1, 2014 and Dec. 22, 2014 from the pathological information 19 of the case ID of P003, and writes the information to the data item of the case ID of P003 in the clinical onset table 61.

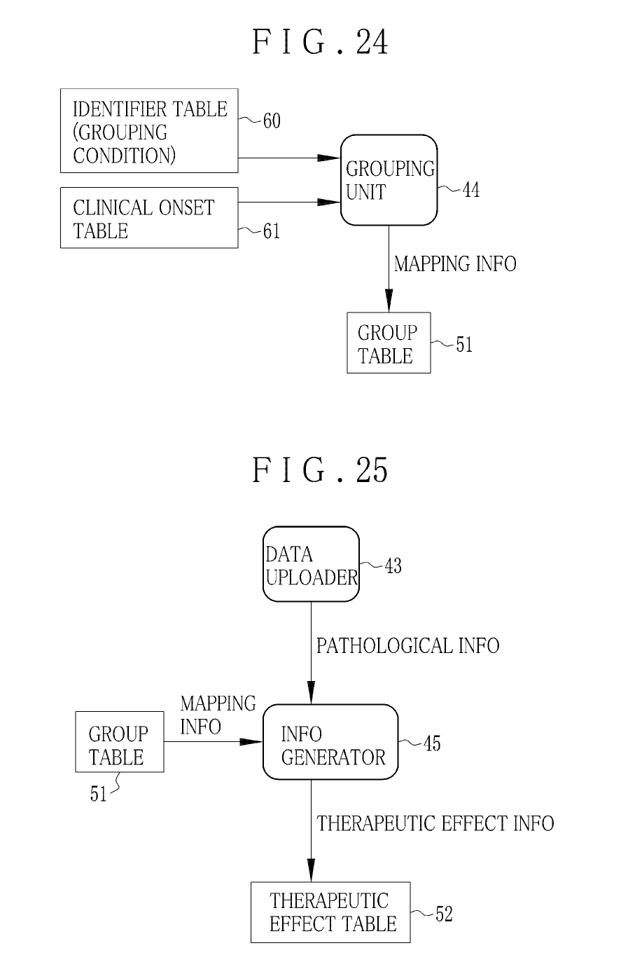

In FIG. 24, the grouping unit 44 groups the lesions for the respective patient bodies according to the identifier table 60 and the clinical onset table 61, and creates mapping information (grouping information). In FIG. 25, the information generator 45 creates therapeutic effect information according to the pathological information 19 from the data uploader 43 and the mapping information from the group table 51.

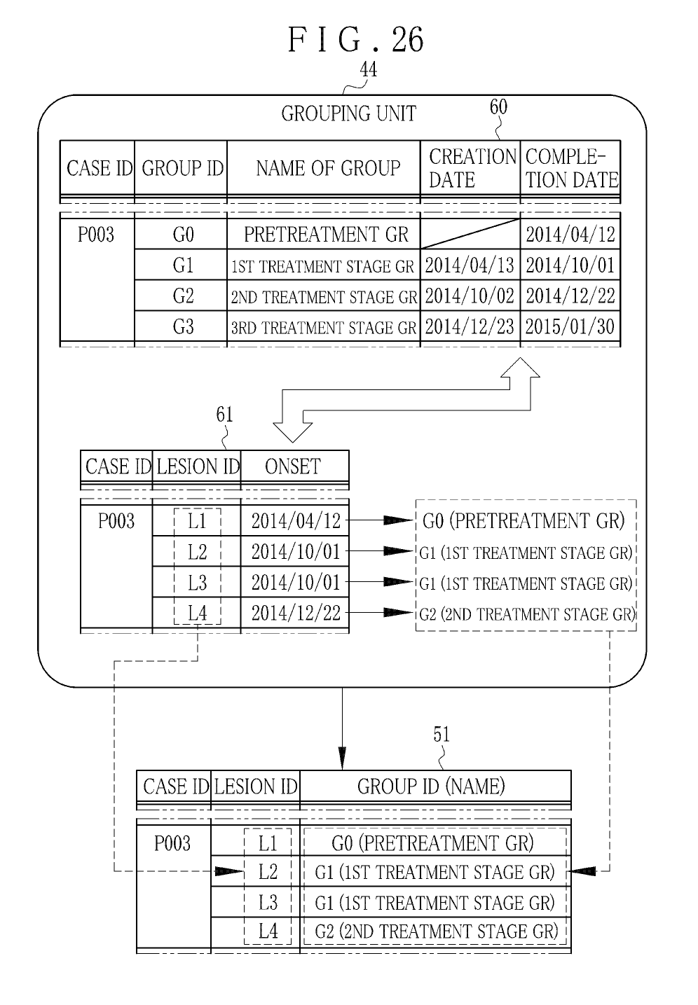

In FIG. 26, creation of mapping information of the patient body of the case ID of P003 is illustrated. The grouping unit 44 checks the creation date and completion date of the respective case groups of the patient body of the case ID of P003 in the identifier table 60 and the clinical onsets of the respective lesion IDs of the patient body of the case ID of P003 in the clinical onset table 61. One of the case groups of which the clinical onset corresponds to the period defined by the creation date and completion date is determined as the case group of the lesion of the lesion ID. For example, the clinical onset of the lesion of the lesion ID of L4 is Dec. 22, 2014. It is judged that a group of this lesion is the second treatment stage group after starting the second therapy session and in a period from the creation date of Oct. 2, 1014 to the completion date of Dec. 22, 2014. See FIG. 22. A group ID of the group determined for each of the lesion IDs is obtained together with the lesion ID as the mapping information, and registered in the group table 51.

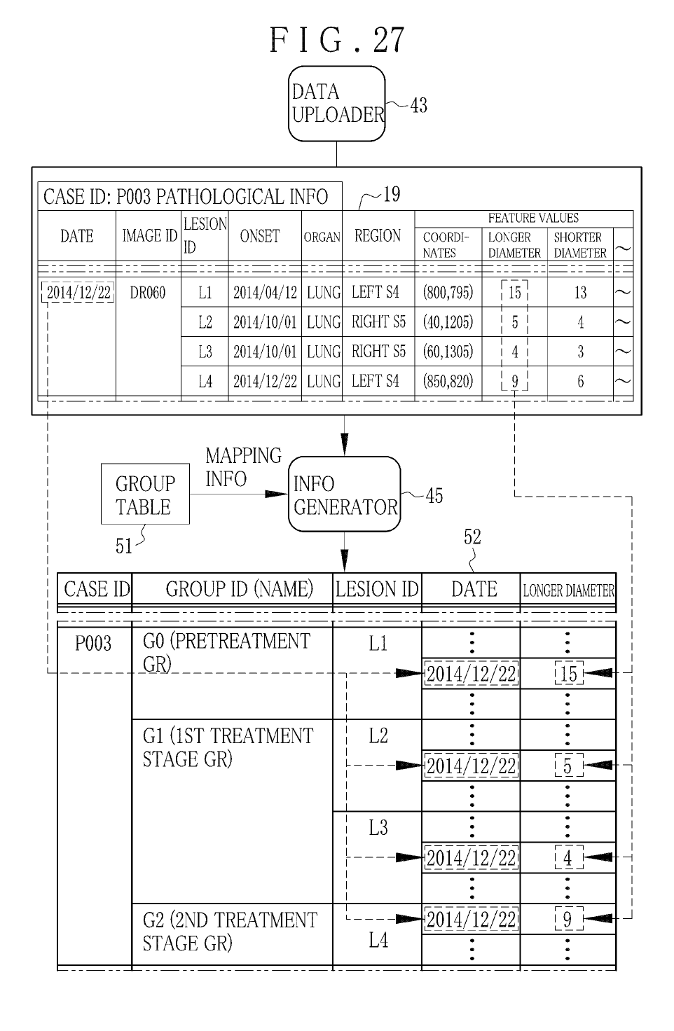

In FIG. 27, creation of the therapeutic effect information of the patient body of the case ID of P003 is illustrated. The information generator 45 reads the date and longer diameter from the pathological information 19 of the patient body of the case ID of P003. The date and longer diameter is written to the data item of the respective lesion IDs of the groups after grouping according to the mapping information of the group table 51 for the case ID of P003 of the therapeutic effect table 52.

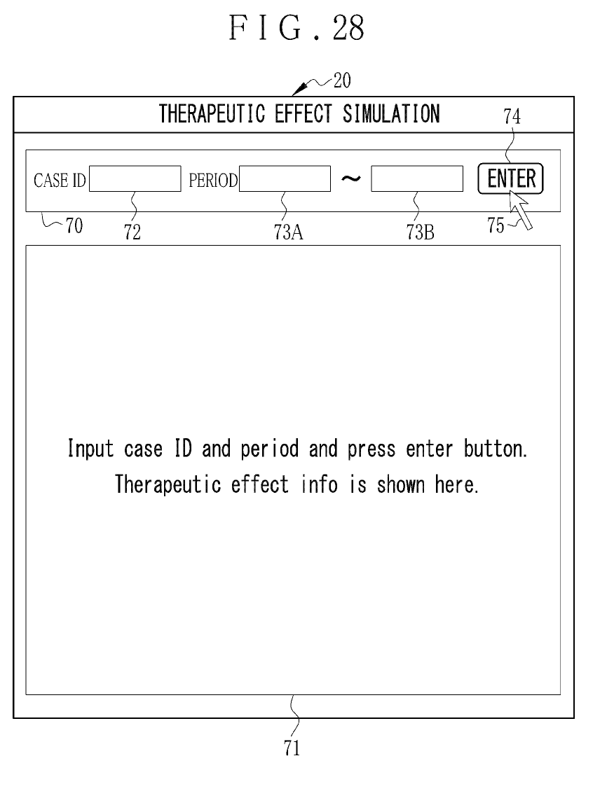

In FIG. 28, the page view 20 includes a first area or input area 70, and a second area or view area 71. The input area 70 is for inputting a request for information distribution of the page view 20. The view area 71 displays therapeutic effect information.

The input area 70 includes an ID input field 72 or input box, period input fields 73A and 73B or input boxes, and a view button 74 or enter button. The ID input field 72 is used for inputting a case ID of a patient body for displaying therapeutic effect information in the view area 71. The period input fields 73A and 73B are used for inputting a period of the therapeutic effect information for display in the view area 71.

In FIG. 28, the page view 20 displayed initially on the display panel 29A after authorization of the medical support apparatus 12 is illustrated. The view area 71 does not display therapeutic effect information, but displays message information for encouraging entry of a case ID and period and selection of the view button 74.

A case ID is input to the ID input field 72. A period is input to the period input fields 73A and 73B. A cursor 75 is operated to press (select) the view button 74. The GUI control unit 35 inputs a request for information distribution to the browser control unit 36 inclusive of the case ID and period at the ID input field 72 and the period input fields 73A and 73B. The browser control unit 36 generates a request for information distribution for which the case ID and the period are queries.

The page generator 47 receives therapeutic effect information from the manager 46 according to the case ID designated as a query in the request for information distribution. The page generator 47 creates graph lines expressing changes of the longer diameter in the time sequence for a date in the period designated as the query in the request among data of the received therapeutic effect information. The page generator 47 causes the view area 71 to display the graph lines as therapeutic effect information.

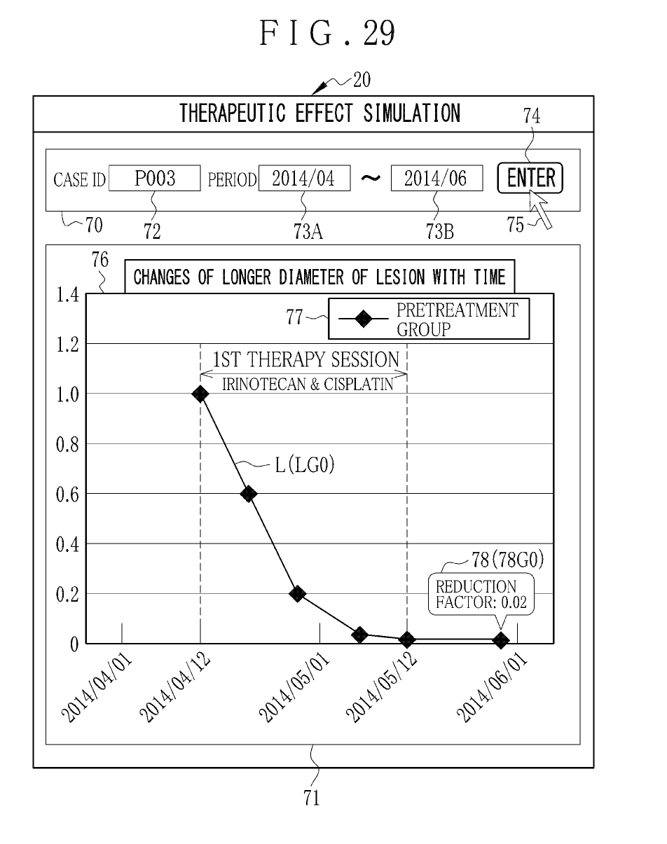

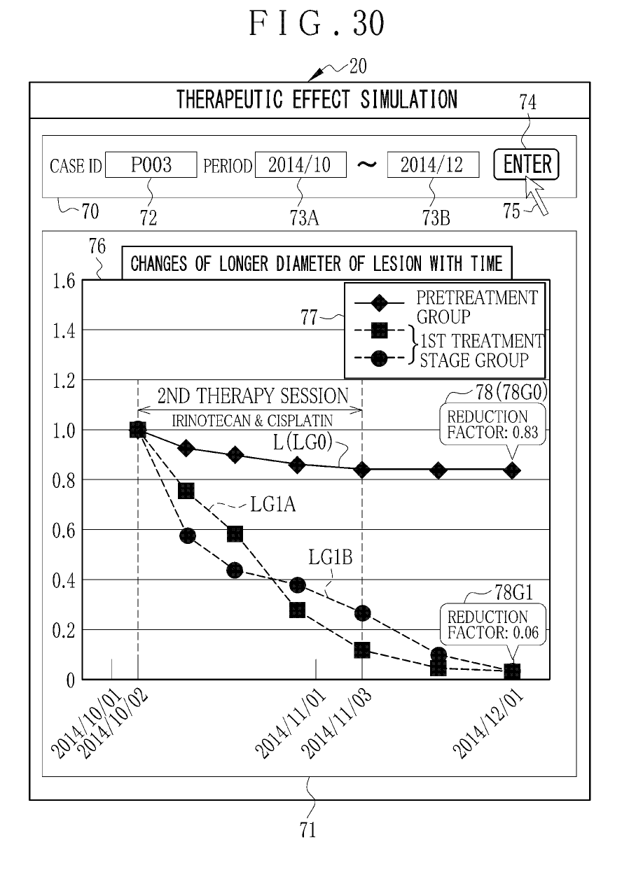

In FIGS. 29 and 30, a two-dimensional plane 76 is formed in the view area 71 by control of the page generator 47, and is defined by a horizontal line for the date and a vertical line for a longer diameter of the form of the lesion. A graph line L is displayed on the two-dimensional plane 76. The graph line L is formed by plotting points from a length of the longer diameter of the respective dates in the two-dimensional plane 76.

An item description 77 and a reduction factor 78 of a lesion are indicated on the two-dimensional plane 76. The item description 77 expresses one of the groups of which the graph line L indicates the change of the longer diameter of the lesion in the time sequence. The item description 77 is indica ted according to the group ID of the therapeutic effect information. The reduction factor 78 is a value obtained by dividing a longer diameter of a newest date in the designated period by a longer diameter of an earliest date in the designated period. For example, the longer diameter of the newest date in the designated period is 1. The earliest diameter of the newest date in the designated period is 41. The reduction factor 78 is 1/41, or approximately 0.02. The therapeutic effect is higher according to the smallness in the reduction factor 78.

The two-dimensional plane 76 displays a period of the anticancer chemotherapy and a name of the anticancer drug according to the treatment history information of the designated case ID.

The longer diameter along the vertical axis is a normalized value of a longer diameter of a certain date with reference to a reference value of 1.0 as a longer diameter of the earliest date in the designated period. Thus, all of the graph lines L are defined with reference to an original point of the longer diameter of 1.0. Also, the longer diameter of the vertical axis of plotting of the graph line L expresses the reduction factor of the longer diameter from the earliest date in the designated period. Note that the longer diameter of the vertical axis may not be normalized. However, normalizing the longer diameter is preferable because the comparison of the graph lines L can be performed more easily.

In FIG. 29, the page view 20 is displayed in order to check therapeutic effect of the first therapy session for the patient body of the case ID of P003. The case ID of P003 is input to the ID input field 72. Values of April 2014 and June 2014 are input to the period input fields 73A and 73B. The view button 74 is selected (pressed). There is no occurrence of the lesions of the lesion IDs of L2-L4 in the period from April 2014 to June 2014, according to the information in the therapeutic effect table 52 in FIG. 14. The therapeutic effect table 52 does not contain this therapeutic effect information. Thus, only a graph line LG0 (indicated by the solid lines and rhombuses) is displayed to indicate a change with time of the longer diameter of the lesion of the lesion ID of L1, which the therapeutic effect information is provided in the period from April 2014 to June 2014. Also, a reduction factor 78G0 of the longer diameter of the lesion of the lesion ID of L1 is indicated by way of the reduction factor 78.

In FIG. 30, the page view 20 is displayed in order to check therapeutic effect of the second therapy session for the patient body of the case ID of P003. The case ID of P003 is input to the ID input field 72. Values of October 2014 and December 2014 are input to the period input fields 73A and 73B. The view button 74 is selected (pressed). There is occurrence of the lesions of the lesion IDs of L2 and L3 in the period from October 2014 to December 2014 in addition to the lesion of the lesion ID of L1, according to the information in the therapeutic effect table 52 in FIG. 14. The therapeutic effect table 52 contains this therapeutic effect information. Thus, a graph line LG1A (indicated by the broken lines and squares) is displayed to indicate a change with time of the longer diameter of the lesion of the lesion ID of L2, and a graph line LG1B (indicated by the broken lines and circles) is displayed to indicate a change with time of the longer diameter of the lesion of the lesion ID of L3. Also, an average reduction factor 78G1 of two reduction factors of the longer diameters of the lesions of the lesion IDs of L2 and L3 is indicated by way of the reduction factor 78, in addition the reduction factor 78G0.

The graph line LG0 expresses changes of the longer diameter of the lesion of the lesion ID of L1 in the pretreatment group in the time sequence. The graph lines LG1A and LG1B express changes of the longer diameters of the lesions of the lesion IDs of L2 and L3 in the first treatment stage group in the time sequence. The reduction factor 78G0 expresses a reduction factor of the longer diameter of the lesion of the lesion ID of L1 in the pretreatment group. The average reduction factor 78G1 expresses an average reduction factor of the longer diameters of the lesions of the lesion IDs of L2 and L3 in the first treatment stage group. Thus, the view area 71 displays the changes of the longer diameters of the lesions of the groups and the reduction factors in a comparable manner.

Also, assuming that the period from December 2014 to February 2015 including the period of the third therapy session is designated (not shown), a graph line L for the change of the longer diameter of the lesion of the lesion ID of L4 in the second treatment stage group after starting the second therapy session is displayed in addition to the graph lines LG0, LG1A and LG1B. Also, the reduction factor 78 of the longer diameter of the lesion of the lesion ID of L4 in the second treatment stage group after starting the second therapy session is displayed in addition to the reduction factors 78G0 and 78G1.

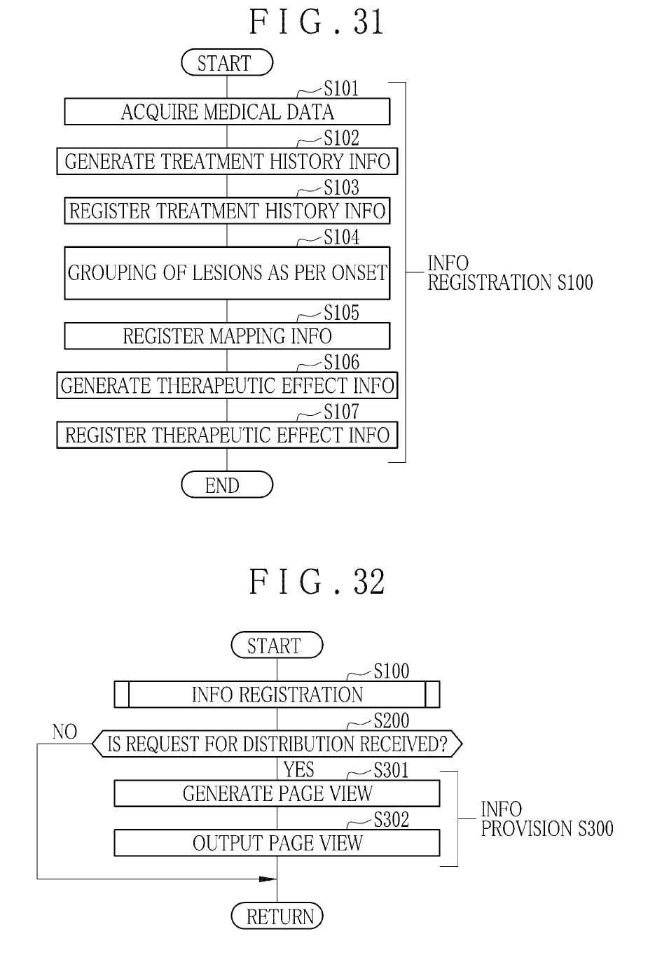

The operation of the above construction is described now by referring to flow charts of FIGS. 31 and 32. In the medical support apparatus 12, information registration S100 is performed in FIG. 31. At first, medical data (clinical data) is acquired from the server cluster 16 by the data uploader 43 in a step S101. The medical data is output to the grouping unit 44 and the information generator 45.

The information generator 45 creates treatment history information according to the medical data of the drug administration and patient event from the data uploader 43 in a step S102. The manager 46 writes (registers) the treatment history information to the treatment history table 50 in a step S103.

The grouping unit 44 creates the identifier table 60 according to the history information from the treatment history table 50 and the identifier list (grouping condition) from the identifier table 49. Also, the clinical onset table 61 is created according to the pathological information 19 from the data uploader 43. The lesions of the respective patient bodies are grouped into groups according to clinical onsets according to the identifier table 60 and the clinical onset table 61 in a step S104. Mapping information (grouping information) is created, and registered to the group table 51 by the manager 46 in a step S105.

Then the information generator 45 creates the therapeutic effect information according to the pathological information 19 from the data uploader 43 and the identifier list from the group table 51 in a step S106. The manager 46 writes (registers) the therapeutic effect information to the therapeutic effect table 52 in a step S107.

The various data are associated with one another by use of the case ID, including information of the anticancer drugs in the treatment history table 50, the lesion IDs and the group IDs in the group table 51, and the therapeutic effect information in the therapeutic effect table 52. It is possible to grasp information of the type of the drug for each patient body, information of the onset and the number of lesions, and information of the therapeutic effect after the anticancer chemotherapy.

To check the therapeutic effect of the anticancer chemotherapy, the doctor performs access to the medical support apparatus 12 with the client terminal apparatus 11, and becomes connected by establishment of authorization. Then the display panel 29A of the client terminal apparatus 11 displays the page view 20 of FIG. 28. He or she inputs a request for information distribution by use of the input area 70 in the page view 20. The client terminal apparatus 11 transmits the request to the medical support apparatus 12 for the page view 20.

In FIG. 32, the request processor 41 in the medical support apparatus 12 receives the request for information distribution (yes in a step S200). The request is input to the page generator 47. Information notification S300 or information provision for therapeutic effect information is performed by the information provider 42. To this end, the therapeutic effect information for the patient body of the case ID designated by the request for the information distribution is sent from the manager 46 to the page generator 47. The page generator 47 creates the page view 20 according to the therapeutic effect information in a step S301. The page view 20 is output to the client terminal apparatus 11 as a requester of the request by the output control unit 48 in a step S302.

In the client terminal apparatus 11, the browser control unit 36 receives the page view 20 from the medical support apparatus 12. The GUI control unit 35 drives the display panel 29A to display the page view 20.

The doctor views the page view 20 and recognizes therapeutic effect of the anticancer chemotherapy. In the view area 71, the graph line L and the reduction factor 78 are displayed as therapeutic effect information, the graph line L indicating changes of the longer diameter of the lesion with time, the reduction factor 78 being for the longer diameter of the lesion within a designated period. He or she can easily check the extent of reduction of the lesion after the anticancer chemotherapy. For example, the lesion in FIG. 29 has become extremely small before disappearance after the anticancer chemotherapy of the first therapy session, so that good therapeutic effect is obtained by the first therapy session of the anticancer chemotherapy.

The grouping unit 44 groups the lesions into groups according to the clinical onsets. The information generator 45 creates the therapeutic effect information for the respective groups. Thus, changes of the longer diameter of the lesions of the groups and their reduction factors can be arranged and indicated in a comparable manner graphically with the graph lines LG0, LG1A and LG1B and the reduction factors 78G0 and 78G1 in FIG. 30. A doctor or operator can easily view the therapeutic effect in a distinct manner between the groups.

It is understood from the graph lines LG1A and LG1B and the average reduction factor 78G1 in FIG. 30 that therapeutic effect is obtained for the lesion in the treatment stage group in the second therapy session in the same manner as the lesion in the pretreatment group in the first therapy session. However, it is understood from the graph line LG0 and the reduction factor 78G0 that only small therapeutic effect is obtained for the lesion of the pretreatment group in the second therapy session in comparison with the lesion in the first treatment stage group.

The clarification in differences in the therapeutic effect between clinical onsets of the lesions makes it possible for the doctor easily to decide the treatment plan of the anticancer chemotherapy, for example, a succeeding therapy session can be based on targeting a lesion of a group of a relatively lower therapeutic effect. Assistance of the support system to the doctor who must decide the treatment plan in the anticancer chemotherapy can be effective considerably.

The grouping is performed by referring to the starting date of each therapy session of the anticancer chemotherapy. It is possible to clarify a difference in the therapeutic effect between the lesion of the onset before the start of each therapy session of the anticancer chemotherapy and the lesion of the onset after the start of each therapy session of the anticancer chemotherapy.

Grouping can be performed into a pretreatment group with an original lesion of which a clinical onset is earlier than the starting date of the first therapy session, and a treatment stage group with a secondary lesion of which a clinical onset is at the same time as or later than the starting date of the first therapy session. Thus, the difference in the therapeutic effect between the original lesion and the secondary lesion can be clarified for indication.

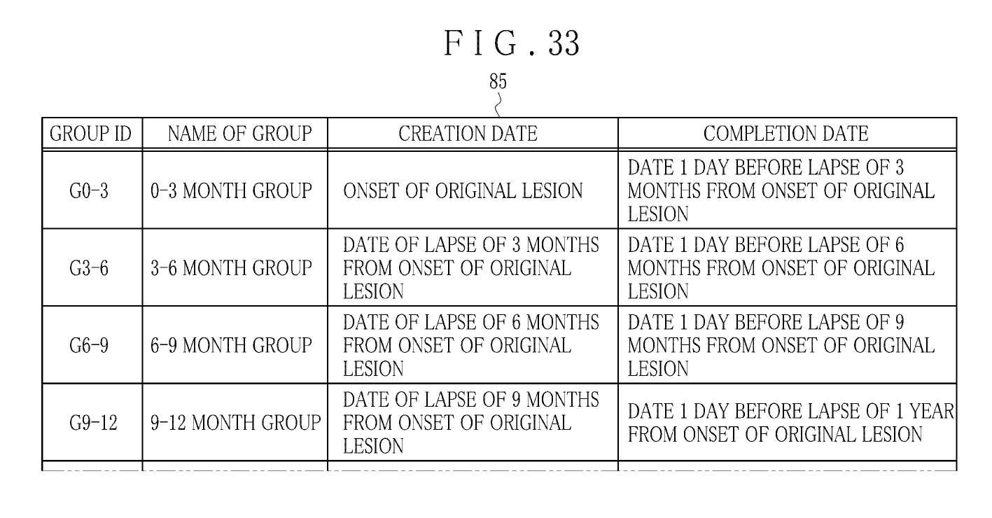

Note that grouping for plural groups can be performed with reference to a clinical onset of an original lesion as indicated in an identifier table 85 (grouping condition table) of FIG. 33.

In FIG. 33, information in the identifier table 85 includes identifier lists (grouping conditions) of plural groups defined by unit periods of three months after the clinical onset of the original lesion. For example, a first group of a group ID of G0-3 is a group of a period from the clinical onset of the original lesion until a date one day before the lapse of three months from the clinical onset. A second group of a group ID of G3-6 is a group of a period from the date of the lapse of three months until a date one day before the lapse of sixth months from the clinical onset. Thus, lesions can be grouped even assuming that a starting date of a respective therapy session is unknown, because the groups are defined irrespective of starting dates of respective therapy session. Furthermore, it is possible to set periods variable for defining case groups according to a manual input of a doctor or operator by use of the page view 20, for example, by a unit period of one month, three months, six months or the like.

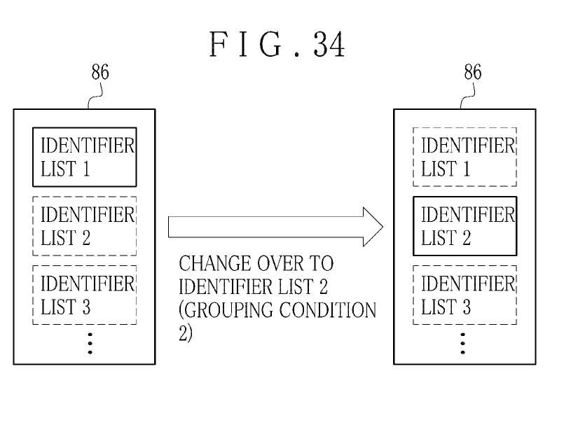

In FIG. 34, an example of manually changing an identifier list by a doctor or operator is illustrated schematically. An identifier table 86 (grouping condition table) is prepared, and contains the plural identifier lists 1, 2, 3 and so on, including those of FIGS. 10 and 33. One of the identifier lists for use is changed over by the manipulation according to requirements of the doctor. In FIG. 34, changeover from the identifier list 1 on the left side to the identifier list 2 on the right side is illustrated.

Furthermore, the identifier list determined by a doctor with his or her preferences can be registered to an identifier table (grouping condition table).

In the first embodiment, the clinical onset of the lesion is the date of performing the imaging. However, it is likely that the clinical onset of the lesion is not simultaneous with the date of performing the imaging. For example, assuming that the lesion in the diagnostic images 18 on the date of the first medical care is considerably large, occurrence of the lesion is earlier than the first medical care. Furthermore, it is likely that the lesion discovered in the diagnostic images 18 is found to be present in the past diagnostic image 18 according to review of the history. Thus, it is possible in the embodiment to constitute the system to receive manual correction of the recorded clinical onset of the lesion from a doctor, to correct the recorded clinical onset.

In the first embodiment, the medical data is acquired by the data uploader 43 periodically, so as to group the lesions in the grouping unit 44 and to create therapeutic effect information in the information generator 45. However, receiving a request for information distribution in the request processor 41 can be utilized for acquiring medical data, grouping the lesions and creating therapeutic effect information.

### Second Embodiment

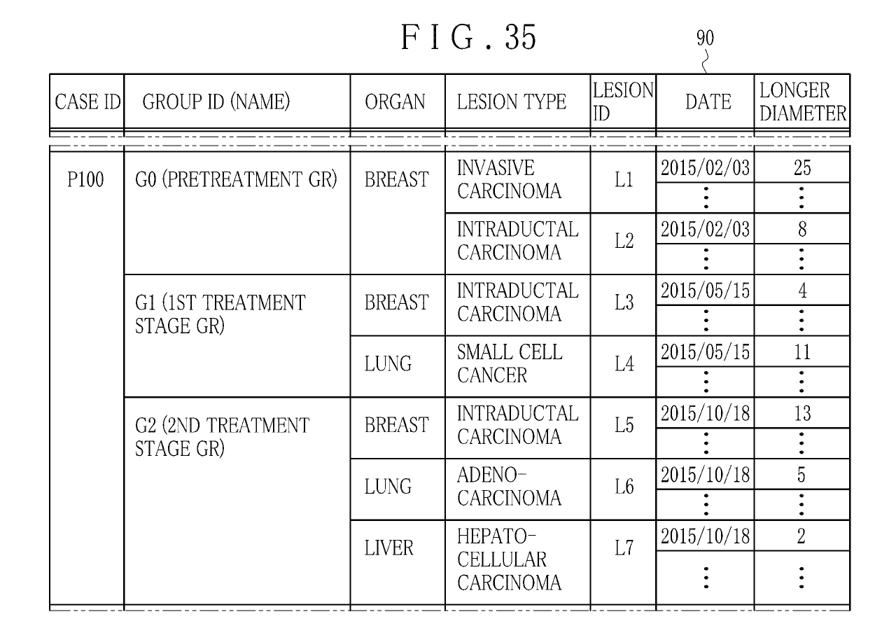

The therapeutic effect of the anticancer chemotherapy may differ according to a body organ of interest (body part) of occurrence of a lesion, or a lesion type of the lesion. In a second preferred embodiment of FIG. 35, the lesions grouped according to the clinical onsets are further grouped (sub-grouped) according to organs of interest and lesion types. To this end, information of the lesion type is recorded with each of the lesion IDs in the pathological information 19. It is possible to record the lesion type according to a manual input of a doctor, or according to automatic recognition in the image server apparatus 15 by image analysis.

In FIG. 35, a therapeutic effect table 90 of the embodiment contains data items of organs of interest and lesion types. The lesions in the respective groups are further grouped (sub-grouped) according to the organs of interest and lesion types. For example, lesions grouped in the first treatment stage group after starting the first therapy session include a lesion of an intraductal carcinoma in a breast with a lesion ID of L3 and a lesion of a small cell cancer in a lung with a lesion ID of L4.

In short, the lesions are grouped according to the organs of interest and the lesion types. The graph line L can be indicated in a grouped manner between the organs of interest and the lesion types, and the reduction factor 78 can be displayed for respectively the organs of interest and lesion types. Note that it is possible to group the lesions according to only either one of the organs of interest and the lesion types, instead of both of the organs of interest and the lesion types in FIG. 35.

Also, it is possible to display therapeutic effect information as a result of statistical processing of therapeutic effect information registered in the therapeutic effect table 90 by the unit of the groups, organs of interest and lesion types.

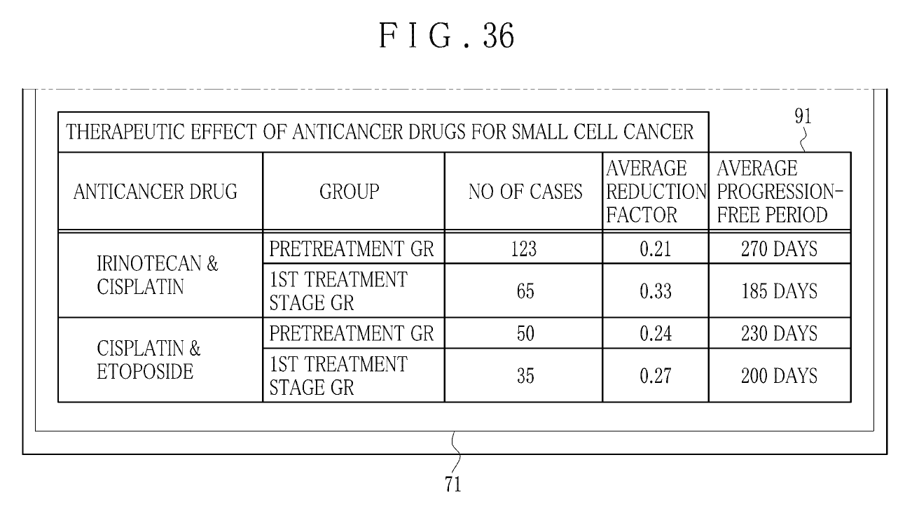

For example, a drug therapeutic effect list 91 of FIG. 36 is displayed in the view area 71 of the page view 20 for therapeutic effect of the respective anticancer drugs in relation to the small cell cancer in the lung by way of the therapeutic effect information. The drug therapeutic effect list 91 contains information of the number of the cases (patients) in the pretreatment group and the respective treatment stage groups, averages of the reduction factors and progression-free periods (average reduction factors and average progression-free periods) for lesions of the small cell cancer in each one of the pretreatment group and the respective treatment stage groups, for combinations of the anticancer drugs “irinotecan and cisplatin” and “cisplatin and etoposide”.

Information of the anticancer drugs can be obtained from the treatment history table 50. The number of the cases can be obtained by counting the patient bodies in which the anticancer drugs of the respective combinations have been used. A method of obtaining an average reduction factor is as follows. A minimum of the longer diameter included in the therapeutic effect information is divided by its maximum to obtain reduction factors of the plural cases. The reduction factors of the cases are added up to obtain a sum, which is divided by the number of the cases to obtain the average reduction factor.

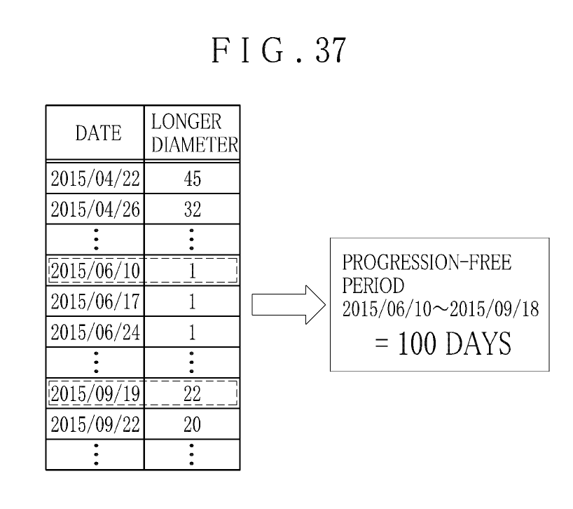

Note that the progression-free period is a period from stabilization to exacerbation of the lesion in the course of the anticancer chemotherapy. In FIG. 37, let a lesion of a patient be reduced by the anticancer chemotherapy. The lesion comes to have a longer diameter of “1” on the date of Jun. 6, 2015, and then have a longer diameter of “22” on the date of Sep. 19, 2015 after recurrence. The progression-free period is 100 days from Jun. 6, 2015 to Sep. 18, 2015. The effect is high according to a length of the progression-free period. An average progression-free period can be obtained by adding up progression-free periods of the respective cases and dividing the sum by the number of the cases, in a manner similar to the average reduction factor.

Consequently, the doctor can decide his or her treatment plan for anticancer chemotherapy suitably by use of the displayed therapeutic effect information as a result of statistical processing. For example, therapeutic effect of respective anticancer drugs to anticancer chemotherapies in the groups can be clearly indicated in the drug therapeutic effect list 91 of FIG. 36. Therapeutic effect for a lesion in the pretreatment group is higher in the use of the combined anticancer drugs of “irinotecan and cisplatin”. However, therapeutic effect for a lesion in the first treatment stage group after starting the first therapy session is higher in the use of the combined anticancer drugs of “cisplatin and etoposide”. It is found that the selection of the anticancer drugs of “cisplatin and etoposide” is more recommendable than selection of the anticancer drugs of the “irinotecan and cisplatin” for the purpose of specific treatment of the lesion in the first treatment stage group after starting the first therapy session.

In the first embodiment, a change of the longer diameter of the lesion with time is indicated in a form of the graph line L. However, the change with time can be indicated in a form of a list in which dates and longer diameters of the lesion are arranged. Furthermore, other data can be displayed as the therapeutic effect information, including a change of a shorter diameter of the lesion with time, a reduction factor, and the like. Other data can be displayed as the therapeutic effect information in the use of statistical processing, such as a survival rate, a recurrence rate, a response rate and the like. The survival rate is a rate of survival of patients upon lapse of a predetermined number of years from the start of the anticancer chemotherapy, for example, five years. The recurrence rate is a rate of recurrence of a disease of patients, for example, upon lapse of one year. The response rate is a rate of patients of complete disappearance of a lesion, or patients of reduction of a lesion in a predetermined proportion.

### Third Embodiment

Search of similar images is frequently performed in the course of treatment of patients. Among plural diagnostic images 18 of the same symptom as the patient, a common symptom image 18S or reference image is searched with a lesion having a similar lesion to a lesion in a patient image 18R of the patient of interest.

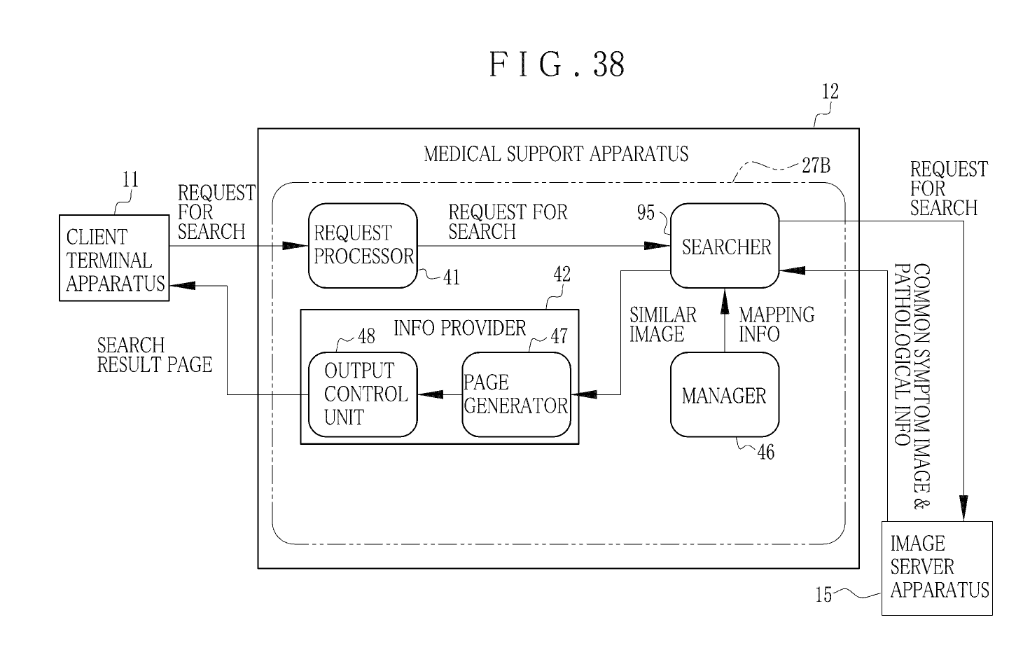

In the similar image search of the known technique, similarity according to the image feature values of the lesion in the patient image 18R and the lesion in the common symptom image 18S is considered. However, similarity of the groups to which the lesions are mapped is not considered. In FIGS. 38-41, a third preferred embodiment is illustrated. The CPU 27B of the medical support apparatus 12 has a function of similar image search, which is performed also in consideration of the groups to which the lesions are mapped.

In FIG. 38, a searcher 95 (search requester) is established in the CPU 27B of the medical support apparatus 12 in the embodiment in addition to the circuit devices included in the first embodiment. Note that the data uploader 43, the grouping unit 44 and the information generator 45 are omitted from depiction in FIG. 38.

The request processor 41 receives a request for search of a similar image from the client terminal apparatus 11. The request processor 41 inputs the request for the search to the searcher 95.

The request for the search includes the image ID of the patient image 18R, and also specific information for specifying the same symptom as the patient of interest. Examples of the specific information include information of a body organ, lesion type and the like of the lesion of the patient of interest.

The searcher 95 sends a request for search to the image server apparatus 15. The image server apparatus 15 searches the common symptom image 18S among the plural diagnostic images 18 in the image database 15A in a condition of the same modality (test type) and the same region of interest as the patient image 18R, and the same organ and the same lesion type as designated by the request for search according to the pathological information 19. The image server apparatus 15 transmits the common symptom image 18S being obtained and the pathological information 19 to the searcher 95. The searcher 95 acquires the common symptom image 18S and the pathological information 19 from the image server apparatus 15.

The searcher 95 performs fine selection of the common symptom image 18S as a final decision among the received candidates of the common symptom image 18S from the image server apparatus 15. To this end, the searcher 95 determines a similarity score in the lesion between the patient image 18R and the common symptom image 18S.

An example of the similarity score is disclosed in JP-A 2011-118543, and is a sum of products of a suitable weighting coefficient and an absolute value of a difference in an image feature value between the lesion in the patient image 18R and the lesion in the common symptom image 18S. Assuming that the difference in the image feature value is smaller, the similarity score is smaller, so that similarity is high between the lesions in the patient image 18R and the common symptom image 18S. Assuming that the difference in the image feature value is larger, the similarity score is larger, so that similarity is low between the lesions in the patient image 18R and the common symptom image 18S. Note that the image feature values are recorded and included in the pathological information 19 from the image server apparatus 15.

The searcher 95 determines a threshold for the similarity score, and judges that the common symptom image 18S is a similar image by checking satisfaction of the checking condition with the threshold. The searcher 95 outputs the common symptom image 18S to the page generator 47 after judgment of the similar image.

The page generator 47 creates a search result page (not shown) for displaying the patient image 18R and the common symptom image 18S output from the searcher 95 as a similar image in a comparable manner. The output control unit 48 transmits the search result page to the client terminal apparatus 11 as a requester of the request. The search result page is output in a data format of display data for web distribution in the same manner as the page view 20.

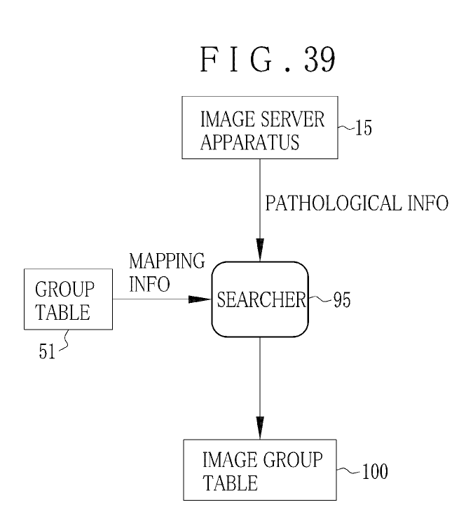

In FIG. 39, the searcher 95 creates an image group table 100 according to the pathological information 19 of the common symptom image 18S from the image server apparatus 15 and the mapping information from the group table 51 before determining the similar image. The image group table 100 contains a registered group to which a lesion in the common symptom image 18S is mapped for respectively the common symptom image 18S received from the image server apparatus 15.

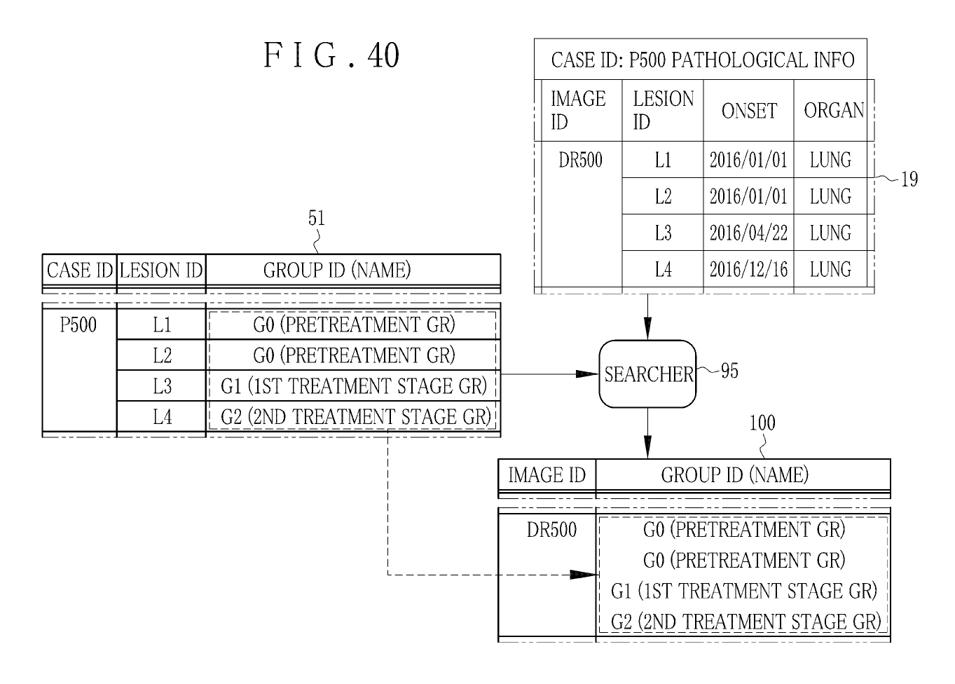

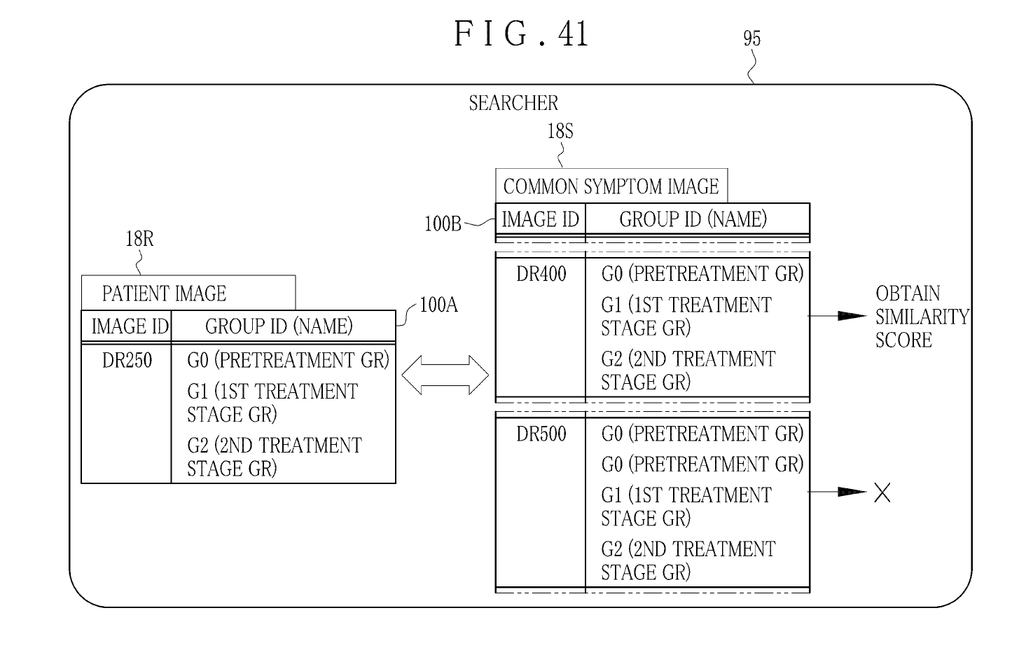

In FIG. 40, creation of the image group table 100 for the common symptom image 18S corresponding to the image ID of DR500 of the case ID of P500 is illustrated. The searcher 95 considers the mapping information of the case ID of P500 in the group table 51, and checks in which of the groups the lesion IDs of L1, L2, L3 and L4 recorded with the image ID of DR500 in the pathological information 19 of the case ID of P500 are grouped by the grouping unit 44. A group ID of a detected one of the groups is written for a data item of the image ID of DR500 in the image group table 100. Note that in relation to the common symptom image 18S and the patient image 18R other than those of the image ID of DR500, the searcher 95 similarly creates the image group table 100 by checking a group of lesions with the mapping information.

The searcher 95 compares the group IDs of the patient image 18R and of the common symptom image 18S registered in the image group table 100, and selectively reads out the common symptom image 18S of which the group IDs of the same combination as the patient image 18R are registered. Only the common symptom image 18S being read out is a target of obtaining the similarity score.

In FIG. 41, search of a similar image of the patient image 18R of the image ID of DR250 is illustrated. The searcher 95 designates the common symptom image 18S of the image ID of DR400 as a target of the similarity score. An image group table 100A for the patient image 18R contains a registered combination of the group IDs of G0, G1 and G2. An image group table 100B for the common symptom image 18S contains the same registered combination of the group IDs of G0, G1 and G2 as the patient image 18R. The common symptom image 18S of the image ID of DR500 is an image of a combination of the group ID different from the patient image 18R. The searcher 95 removes the common symptom image 18S from candidates of the similar images without calculating the similarity score.

Thus, attention to the common symptom image 18S for determining the similarity score as an image of which the group IDs of the same combination as the patient image 18R is registered makes it possible to detect the similar image from the common symptom image 18S with a lesion in the same group as a lesion in the patient image 18R. It is possible to provide a similar image in a condition of high similarity score for the group of the lesion in addition to the high similarity score according to the image feature value.

There are various methods of searching similar images in consideration of the group to which the lesion is mapped. Two of the methods are described below, in addition to the method in which only the common symptom image 18S of the registered group IDs of the same combination as the patient image 18R described above is used for obtaining the similarity score.

The first method is to determine a similarity score for any one of the common symptom image 18S transmitted from the image server apparatus 15, and extracts the common symptom image 18S as a candidate of the similar image by evaluation according to the threshold. Among plural candidates of the similar image being read out, the common symptom image 18S of which the group IDs of the same combination as the patient image 18R are selected further, and is output finally as a similar image.

The second method is to set higher probability in determining a similar image for the common symptom image 18S of which the group IDs of the same combination as the patient image 18R are registered than for the common symptom image 18S of which the group IDs of a different combination from the patient image 18R are registered.

For example, the method of JP-A 2011-118543 is used for determining the similarity score. For the common symptom image 18S with the registered group IDs of which the combination is different from that of the patient image 18R, a value of 10 is added to the similarity score. For the common symptom image 18S with the registered group IDs of which the combination is the same as that of the patient image 18R, a value of 10 is subtracted from the similarity score.

Note that the similarity score can be increased or decreased in consideration with the extent of the equality to the patient image 18R in relation to the common group IDs. For example, assuming that there is equality to the patient image 18R for all of the group IDs, 10 is subtracted from the similarity score. Assuming that there is equality to the patient image 18R for only one of the group IDs, 5 is subtracted from the similarity score. Assuming that there is a difference from the patient image 18R for all of the group IDs, 10 is added to the similarity score.

Also, criteria for determining the similarity score can be less strict for the common symptom image 18S with the registered group IDs of which the combination is the same as that of the patient image 18R than for the common symptom image 18S with the registered group IDs of which the combination is different from that of the patient image 18R.

It is preferable in the search result page to display treatment history information of the anticancer chemotherapy performed to the patient body of the similar image, and therapeutic effect information, which includes changes of a size in a time sequence, reduction factor and progression-free period of a lesion in the similar image. It is possible to determine a treatment plan of the anticancer chemotherapy of the patient by referring to the treatment history information and therapeutic effect information of the patient body of the similar image at the same time as the similar image search.

Hardware construction of the computer for constituting the medical support apparatus 12 of the present invention can be modified suitably. For example, the medical support apparatus 12 can be constituted by a plurality of server computers discrete from one another for the purpose of increasing performance of processing and reliability. Specifically, a first server computer may constitute the request processor 41 and the information provider 42. A second server computer may constitute the data uploader 43, the grouping unit 44 and the information generator 45. A third server computer may constitute the manager 46. The medical support apparatus can be constituted by the three server computers. Also, the therapeutic effect database 12A can be combined with the third server computer having the manager 46.

Also, the image server apparatus 15 can function for search performed by the searcher 95 in the third embodiment. To this end, the group table 51 is referred to not only with the therapeutic effect database 12A but with the image database 15A. The image server apparatus 15 constitutes a medical support apparatus of the present invention in combination with the medical support apparatus 12.

Also, the therapeutic effect database 12A may be discrete from the medical support apparatus. Furthermore, a single system apparatus including the medical support apparatus 12, the EMR server apparatus 14 and the image server apparatus 15 can be provided in the present invention. The EMR database 14A and the image database 15A can be combined with the medical support apparatus 12.

In the first embodiment, the medical support apparatus 12 creates the page view 20. The client terminal apparatus 11 drives the display panel 29A to display the page view 20 according to the display data of the page view 20 from the medical support apparatus 12. However, it is possible to transmit therapeutic effect information as an origin of the page view 20 from the medical support apparatus 12 to the client terminal apparatus 11, and to create the page view 20 in the client terminal apparatus 11. The information provider 42 is constituted by an information output control unit for controlling an output of the therapeutic effect information as an origin of the page view 20. The page generator 47 is established in the CPU 27A in the client terminal apparatus 11.

Also, the various circuit devices which are provided in the CPU 27B of the medical support apparatus 12 except for the output control unit 48 may be established in the CPU 27A of the client terminal apparatus 11, which can be operated as a medical support apparatus. The request processor 41 receives a command signal for information distribution from the GUI control unit 35 in place of the request for information distribution. The page generator 47 sends the page view 20 to the GUI control unit 35. The GUI control unit 35 operates with the function of the output control unit 48.

Thus, the construction of the hardware in the computer can be changed suitably according to performance requiring ability for processing, safety, reliability and the like. Additionally, it is also possible to store the computer-executable program 40 and other application programs in a duplicated form or divided form in plural storage media for the purpose of ensuring the safety and the reliability.

The information notification or information provision of the therapeutic effect information is not limited to the above embodiment, in which the page view 20 is distributed on the web. For example, a database for storing a file of the therapeutic effect information is provided. A doctor or operator is authorized to perform access to the database, to read out the file from the database. Also, a well-known file transfer protocol, such as FTPS (File Transfer Protocol over SSL/TLS), can be used to transmit the file to the client terminal apparatus 11 automatically. An electronic mail can be used instead of the file transfer protocol. Also, printed material of a paper sheet or the like can be used for the therapeutic effect information.

In the above embodiments, the medical support apparatus 12 is used in one medical facility. However, it is possible to use the medical support apparatus 12 commonly in a plurality of medical facilities.

In the above embodiments, the client terminal apparatus 11 in one hospital facility is connected to the medical support apparatus 12 communicably by use of the LAN 13 or other network. The medical support apparatus 12 provides the page view 20 of medical support in response to a request from the client terminal apparatus 11. To use the page view 20 in the plural hospital facilities, the medical support apparatus 12 is set on-line with the plural client terminal apparatuses 11 positioned in the hospital facilities by use of the wide area network (WAN), such as the Internet, public communication network and the like. Requests from the client terminal apparatuses 11 of the hospital facilities are received by the medical support apparatus 12 with the WAN, to provide the page view 20 of the medical support to the client terminal apparatus 11. Note that information security should be established for use of the WAN, for example, the Virtual Private Network (VPN) or Hypertext Transfer Protocol Secure (HTTPS) can be preferably used as communication protocol of a high level of security.

The EMRs 17 and the diagnostic images 18 are managed for each one of the medical facilities. A place of installation and manager of the medical support apparatus 12 can be a data center of a service provider (company) separate from the medical facilities, but can be a suitable one of the plural medical facilities.

The present invention is not limited to the above embodiments. Various features of the embodiments and variants of the invention can be combined with each other suitably. Also, the computer-executable program and a storage medium for storing the computer-executable program are included in the scope of the present invention.

According to one embodiment mode of the invention, a non-transitory computer readable medium for storing a computer-executable program is provided, the computer-executable program enabling execution of computer instructions to perform operations for medical support in an anticancer chemotherapy. The operations include grouping plural lesions of a patient body treated by the anticancer chemotherapy into plural groups according to clinical onsets of the lesions. The operations include creating therapeutic effect information of therapeutic effect of the anticancer chemotherapy for respectively the plural groups. The operations include providing the therapeutic effect information.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A medical support apparatus comprising:
a grouping unit for grouping plural lesions of a patient body treated by an anticancer chemotherapy into plural groups according to clinical onsets of said lesions;
an information generator for creating therapeutic effect information of therapeutic effect of said anticancer chemotherapy for respectively said plural groups; and
an information provider for providing said therapeutic effect information.

2. A medical support apparatus as defined in claim 1, wherein assuming that said anticancer chemotherapy for said patient body is performed in plural therapy sessions, said groups are defined with reference to starting dates of respectively said plural therapy sessions.

3. A medical support apparatus as defined in claim 2, wherein said plural groups include:
a pretreatment group for grouping of a lesion of which a clinical onset is earlier than a starting date of a first therapy session among said therapy sessions;
a treatment stage group for grouping of a lesion of which a clinical onset is simultaneous with or later than said first therapy session and in a period from a starting date of a current therapy session until a date one day before a starting date of a therapy session next to said current therapy session.

4. A medical support apparatus as defined in claim 1, wherein said lesions are further grouped according to at least one of a body organ of occurrence of said lesions, and a lesion type of said lesions.

5. A medical support apparatus as defined in claim 1, wherein a condition of grouping of said groups is variable by an input action of manual operation.

6. A medical support apparatus as defined in claim 1, further comprising a manager for managing stored information of said lesions, said groups, an anticancer drug and said therapeutic effect information in an associated manner.

7. A medical support apparatus as defined in claim 1, further comprising a searcher for selectively reading out a common symptom image among plural diagnostic images, wherein said common symptom image is an image of one patient body having a symptom similar to a symptom of said patient body, and is an image of a lesion characteristically similar to a lesion in a patient image of said patient body;
wherein said searcher performs said readout by referring to one of said groups mapped to said lesion in said patient image and one of said groups mapped to said lesion in said common symptom image.

8. A medical support apparatus as defined in claim 1, wherein said therapeutic effect information includes at least one of a change in a size of said lesions with time, a reduction factor of said size of said lesions, and a progression-free period from stabilization of said lesions after said anticancer chemotherapy until exacerbation of said lesions.

9. A medical support apparatus as defined in claim 1, wherein said information provider includes:
a page generator for generating a page view of said therapeutic effect information;
an output control unit for controlling an output of said page view.

10. A method of operating a medical support apparatus comprising steps of:
grouping plural lesions of a patient body treated by an anticancer chemotherapy into plural groups according to clinical onsets of said lesions;
creating therapeutic effect information of therapeutic effect of said anticancer chemotherapy for respectively said plural groups; and
providing said therapeutic effect information.

11. A medical support system including a medical support apparatus, and a client terminal apparatus connected to said medical support apparatus by network connection, comprising:
a grouping unit for grouping plural lesions of a patient body treated by an anticancer chemotherapy into plural groups according to clinical onsets of said lesions;
an information generator for creating therapeutic effect information of therapeutic effect of said anticancer chemotherapy for respectively said plural groups; and
an information provider for providing said therapeutic effect information.
